(19) United States
(12) Patent Application Publication
(10) Pub. No.: US 2006/0251665 A1
(43) Pub. Date: Nov. 9, 2006

(54) Vaccines

(76) Inventors: Michael Neil Burden, King of Prussia, PA (US); Jonathan Henry Ellis, King of Prussia, PA (US); Paul A Hamblin, King of Prussia, PA (US)

Correspondence Address: GlaxoSmithKline;Corporate Intellectual Property UW2220, PO Box 1539, King of Prussia, PA, 19406-0939, US

(21) Appl. No.: 10/515,871

(22) PCT Filed: May 23, 2003
(86) PCT No.: PCT/EP03/05594

§ 371 Date: Mar. 22, 2006

(30) Foreign Application Priority Data
May 24, 2002 (GB) 0212046.7

(51) Int. Cl. A61K 39/00; C07H 21/04; C12P 21/06; A61K 48/00; C07K 14/705

(52) U.S. Cl. 424/185.1; 514/44; 435/69.1; 435/320.1; 435/325; 530/350; 536/23.5

(57) ABSTRACT

The present invention relates to the novel nucleic acid constructs, useful in nucleic acid vaccination protocols for the treatment and prophylaxis of MUC-1 expressing tumors. In particular, the nucleic acid is DNA and the DNA constructs comprise a gene encoding a MUC-1 derivative having less than 10 perfect repeat units. The invention further provides pharmaceutical compositions comprising said constructs, particularly pharmaceutical compositions adapted for particle mediated delivery, methods for producing them, and their use in medicine. Novel proteins encoded by the nucleic acid and pharmaceutical compositions containing them are also provided.

The present invention relates to the novel nucleic acid constructs, useful in nucleic acid vaccination protocols for the treatment and prophylaxis of MUC-1 expressing tumours. In particular, the nucleic acid is DNA and the DNA constructs comprise a gene encoding a MUC-1 derivative having less than 10 perfect repeat units. The invention further provides pharmaceutical compositions comprising said constructs, particularly pharmaceutical compositions adapted for particle mediated delivery, methods for producing them, and their use in medicine. Novel proteins encoded by the nucleic acid and pharmaceutical compositions containing them are also provided.

### BACKGROUND TO THE INVENTION

The epithelial cell mucin MUC-1 (also known as episialin or polymorphic epithelial mucin, PEM) is a large molecular-weight glycoprotein expressed on many epithelial cells. The protein consists of a cytoplasmic tail, a transmembrane domain and a variable number of tandem repeats of a 20 amino acid motif (herein termed the VNTR monomer, it may also be known as the VNTR epitope, or the VNTR repeat) containing a high proportion of proline, serine and threonine residues. The number of repeats is variable due to genetic polymorphism at the MUC-1 locus, and most frequently lies within the range 30-100 (Swallow et al, 1987, Nature 328:82-84). In normal ductal epithelia, the MUC-1 protein is found only on the apical surface of the cell, exposed to the duct lumen (Graham et al, 1996, Cancer Immunol Immunother 42:71-80; Barratt-Boyes et al, 1996, Cancer Inmunol Immunother 43:142-151). One of the most striking features of the MUC-1 molecule is its extensive O-linked glycosylation. There are five putative O-linked glycosylation sites available within each MUC-1 VNTR monomer. According to the numbering system below, these are Thr-4, Ser-10, Thr-11, Ser-19 and Thr-20.

The VNTR can be characterised as typical or perfect repeats having a sequence as set forth below or minor variation from this perfect repeat comprising two to three differences over the 20 amino acids:

The following is the sequence of the perfect repeat.

```
1 2 3 4 5 6 7 8 9 10 11 12 13 14 15 16 17 18 19 20
A P D T R P A P G S T A P P A H G V T S
                E S T
                                                            A
                                                            Q
```

Amino acids that are underlined may be substituted for the amino acid residues shown.

Imperfect repeats have different amino acid substitutions to the consensus sequence above with 55-90% identity at the amino acid level. The four imperfect repeats are shown below, with the substitutions underlined:

| | |
|---|---|
| APDTRPAPGSTAPPAHGVTS | perfect repeat |
| APATEPASGSAATWGQDVTS | imperfect repeat 1 |
| VPVTRPALGSTTPPAHDVTS | imperfect repeat 2 |
| APDNKPAPGSTAPPAHGVTS | imperfect repeat 3 |
| APDNRPALGSTAPPVHNVTS | imperfect repeat 4 |

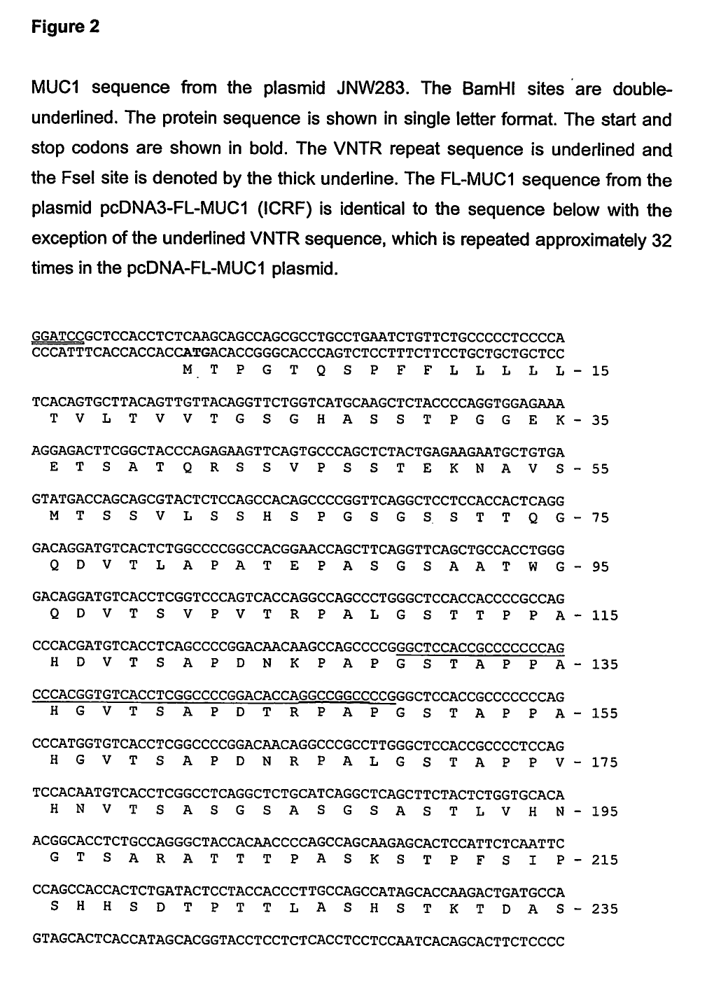

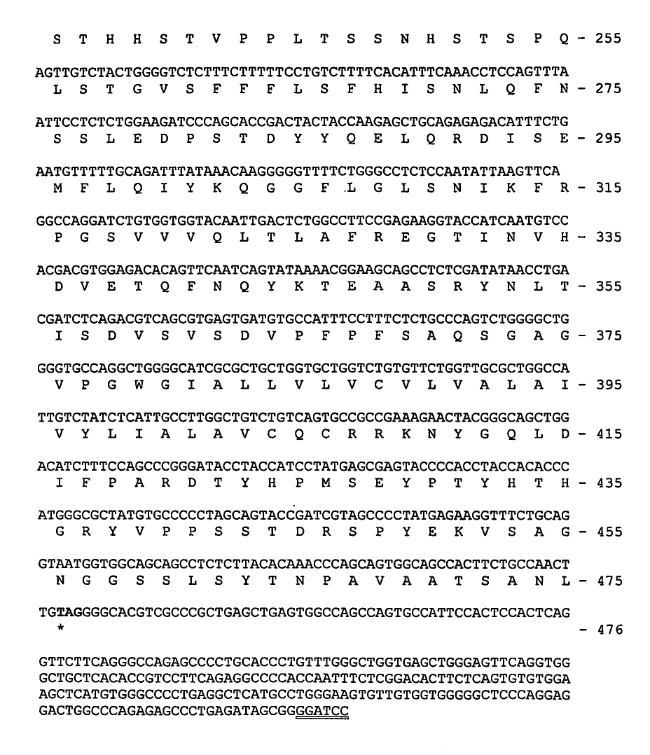

The imperfect repeat in wild type—MUC-1 flank the perfect repeat region. In malignant carcinomas arising by neoplastic transformation of these epithelial cells, several changes affect the expression of MUC-1. The polarised expression of the protein is lost, and it is found spread over the whole surface of the transformed cell. The total amount of MUC-1 is also increased, often by 10-fold or more (Strous & Dekker, 1992, Crit Rev Biochem Mol Biol 27:57-92). Most significantly, the quantity and quality of the O-linked carbohydrate chains changes markedly. Fewer serine and threonine residues are glycosylated. Those carbohydrate chains that are found are abnormally shortened, creating the tumour-associated carbohydrate antigen STn (Lloyd et al, 1996, J Biol Chem, 271:33325-33334). As a result of these glycosylation changes, various epitopes on the peptide chain of MUC-1 which were previously screened by the carbohydrate chains become accessible. One epitope which becomes accessible in this way is formed by the sequence APDTR (Ala 8-Arg 12 in FIG. 2) present in each 20 amino acid VNTR perfect monomer (Burchell et al, 1989, Int J Cancer 44:691-696).

It is apparent that these changes in MUC-1 mean that a vaccine that can activate the immune system against the form of MUC-1 expressed on tumours may be effective against epithelial cell tumours, and indeed other cell types where MUC-1 is found, such as T cell lymphocytes. One of the main effector mechanisms used by the immune system to kill cells expressing abnormal proteins is a cytotoxic T lymphocyte immune response (CTL's) and this response is desirable in a vaccine to treat tumours, as well as an antibody response. A good vaccine will activate all arms of the immune response. However, current carbohydrate and peptide vaccines such as Theratope or BLP25 (Biomira Inc, Edmonton, Canada) preferentially activate one arm of the immune response—a humoral and cellular response respectively, and better vaccine designs are desirable to generate a more balanced response.

Nucleic acid vaccines provide a number of advantages over conventional protein vaccination, in that they are easy to produce in large quantity. Even at small doses they have been reported to induce strong immune responses, and can induce a cytotoxic T lymphocyte immune response as well as an antibody response.

The full-length MUC-1, however, is very difficult to work with due to the highly repetitive sequence, since it is highly susceptible to recombination, such recombination events cause significant biopharmaceutical development difficulties. Additionally the GC rich nature of the VNTR region makes sequencing difficult. Further for regulatory reasons—it is necessary to fully characterise the DNA construct. It is highly problematic to sequence a molecule with such a high frequency repeating structure. Given that it is unknown precisely how many repeat units are in wild type MUC-1 this inability to precisely characterise full-length MUC-1 makes this unacceptable for regulatory approval.

MUC-1 VNTR regions are thought to contain immunodominant epitopes. Surprisingly the present inventors have found that it is possible to reduce the number of VNTRs to produce an immunogenic construct that has equivalent antitumour activity as compares to wild type full-length MUC-1. The construct of the present invention are stable. In particular, the constructs are stable in terms of growth characteristics, plasmid retention and plasmid quality when grown as cultures of E. Coli over the course of 9 passages each lasting between 10-14 hours.

### SUMMARY OF THE INVENTION

The present invention provides a nucleic acid sequence, encoding a MUC-1 antigen, which is capable of raising an immune response in vivo and is stable and has reduced susceptibility to recombination with respect to full-length MUC-1. Stability is a measure of the amount of plasmid in defined form. It is preferred that there is less than 2.0% contamination of recombinogenic forms, as determined on an agarose or polyacryl amide gel, when visualised by the eye, after grown in large scale. Large scale typically means when grown on a greater than one litre scale. It is also a separate measure of stability that plasmid copy remain stable over a period of passages. Preferably the plasmid copy number increases over the number of passages, particularly from passage 1 to 9. Preferably plasmid copy number increase about 10%, 20%, 30%, 35%, 40%, most preferably about 50% over 9 passages. In particular embodiments the invention provides constructs having 1 to 15, preferably between 1 to 10 Perfect VNTR repeat units. It is preferred that there are less than 8 perfect repeats. Preferred embodiments provide DNA constructs with one, two, three, four, five, six and seven repeats respectively. In certain embodiments of the invention, the imperfect repeat region is retained. Preferred are constructs containing one or seven perfect repeats. Proteins encoded by such constructs are novel and form an aspect of the invention.

In further aspect of the invention the nucleic acid sequence is a DNA sequence in the form of a plasmid. Preferably the plasmid is super-coiled.

In a further aspect of the invention there is provided a pharmaceutical composition comprising a nucleic acid sequence as herein described and a pharmaceutical acceptable excipient, diluent or carrier.

Preferably the carrier is a gold bead and the pharmaceutical composition is amenable to delivery by particle mediated drug delivery.

In yet a further embodiment, the invention provides the pharmaceutical composition and nucleic acid constructs for use in medicine. In particular, there is provided a nucleic acid construct of the invention, in the manufacture of a medicament for use in the treatment or prophylaxis of MUC-1 expressing tumours.

The invention further provides for methods of treating a patient suffering from or susceptible to MUC-1 expressing tumour, particularly carcinoma of the breast, lung, ovarian, prostate (particularly non-small cell lung carcinoma), gastric and other GI (gastrointestinal) by the administration of a safe and effective amount of a composition or nucleic acid as herein described.

In yet a further embodiment the invention provides a method of producing a pharmaceutical composition as herein described by admixing a nucleic acid construct or protein of the invention with a pharmaceutically acceptable excipient, diluent or carrier.

### DETAILED DESCRIPTION OF THE INVENTION

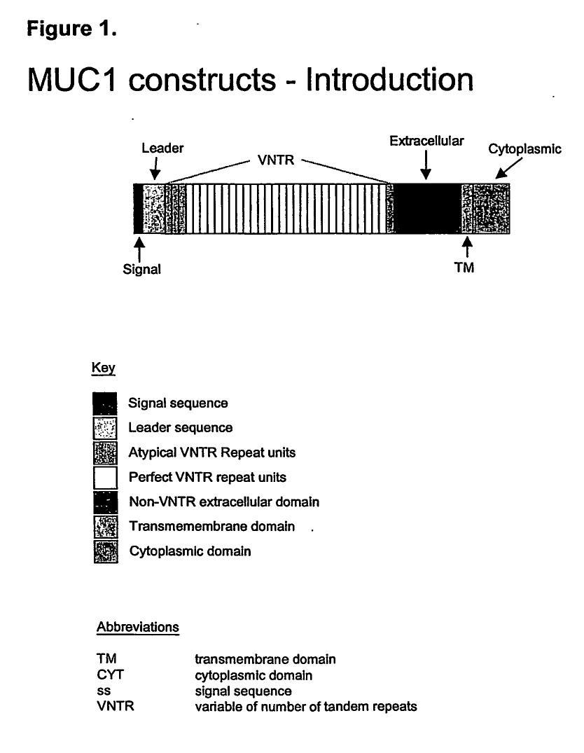

As described herein the nucleic acid constructs of the invention, typically have less than 15, more typically less than 10 perfect repeats. The wild type MUC-1 (See FIG. 1) molecule contains a signal sequence, a leader sequence, imperfect or atypical VNTR, the perfect VNTR region, a further atypical VNTR, a non-VNTR extracellular domain a transmembrane domain and a cytoplasmic domain.

Preferred embodiments of the invention have less than 14, 13, 12, 11, 10, 9, 8,7, 6, 5, 4, 3, 2 repeats. Particularly preferred constructs have 1, 2 or 7 perfect repeats.

The non-VNTR extracellular domain is approximately 80 amino acids, 5′ of VNTR and 190-200 amino acids 3′ VNTR. All constructs of the invention comprise at least one epitope from this region. An epitope is typically formed from at least seven amino acid sequence. Accordingly the constructs of the present invention include at least one epitope from the non VNTR extra-cellular domain. Preferably substantially all or more preferably all of the non-VNTR domain is included. It is particularly preferred that construct contains at least one epitope comprised by the sequence FLSFHISNL, NSSLEDPSIDYYQELQRDISE or NLTISDVSV. More preferred is that two, preferably three epitope sequences are incorporated in the construct.

In a preferred embodiment the constructs comprise an N-terminal leader sequence. The signal sequence, transmembrane domain and cytoplasmic domain are each individually optional in the construct. All may be present, or one or more may be deleted.

Preferred constructs according to the invention are:
1) 7 VNTR MUC-1 (ie Full Muc-1 with only 7 perfect repeats).
2) 7 VNTR MUC-1 Δss (As I, but also devoid of signal sequence)
3) 7 VNTR MUC-1 ΔTM ΔCYT (As 1, but devoid of Transmembrane and cytoplasmic domains)
4) 7 VNTR MUC-1 Δss ΔTM ΔCYT (As 3, but also devoid of signal sequence)

Also preferred are equivalent constructs of 1 to 4 above, but containing only 2 VNTR, or 1 VNTR. The VNTR in such constructs have the sequence of a perfect repeat as herein before described. In an embodiment one or more of the VNTR units is mutated to reduce the potential for glycosylation, by altering a glycosylation site. The mutation is preferably a replacement, but can be an insertion or a deletion. Typically at least one threonine or seriene is substituted with valine, Isoleucine, alanine, asparagine, phenylalanine or tryptophan. In a wild type VNTR monomer there are 5 putative O-linked glycosylation sites available within each MUC-1 VNTR monomer. These are (see numbering above) Thr-4, Ser-10, Thr-11, Ser-19 and Thr-20. It is thus preferred that at least one, preferably 2 or 3 or more, preferably at least four residues are substituted with an amino acid as noted above.

Preferred substitutes include:

| | | |
|---|---|---|
| Thr 4 | → | Val |
| Ser 10 | → | Ala |
| Thr 11 | → | Ile or Val |
| Ser 19 | → | Val |
| Thr 20 | → | Ala |

In a further embodiment the MUC-1 constructs are provided with a nucleic acid sequence encoding a heterologous T-cell epitope. Such epitopes include T-cell epitopes derived from bacterial proteins and toxins, such as Tetanus and Diptheria toxins. For example, the P2 and P30 epitopes from Tetanus toxin. Such epitopes may be part of a longer sequence. The epitopes may be incorporated within the nucleic acid molecule or at the 3′ or 5′ end of the sequence according to the invention.

Other fusion partners may be contemplated such as those derived from Hepatitis B core antigen, or tuberculosis. In an embodiment, a fusion partner derived from Mycobacterium tuberculosis, RA12, a subsequence (amino acids 192 to 323) of MTB32A (Skeiky et al Infection and Immunity (1999) 67: 3998-4007).

Yet other immunological fusion partners, include for example, protein D from Haemophilus influenza B (WO91/18926) or a portion (typically the C-terminal portion) of LYTA derived from Streptococcus pneumoniae (Biotechnology 10: 795-798, 1992).

According to a further aspect of the invention, an expression vector is provided which comprises and is capable of directing the expression of a polynucleotide sequence according to the invention. The vector may be suitable for driving expression of heterologous DNA in bacterial insect or mammalian cells, particularly human cells.

According to a further aspect of the invention, a host cell comprising a polynucleotide sequence according to the invention, or an expression vector according the invention is provided. The host cell may be bacterial, e.g. E. coli, mammalian, e.g. human, or may be an insect cell. Mammalian cells comprising a vector according to the present invention may be cultured cells transfected in vitro or may be transfected in vivo by administration of the vector to the mammal.

The present invention further provides a pharmaceutical composition comprising a polynucleotide sequence according to the invention. Preferably the composition comprises a DNA vector. In preferred embodiments the composition comprises a plurality of particles, preferably gold particles, coated with DNA comprising a vector encoding a polynucleotide sequence of the invention which the sequence encodes a MUC-1 amino acid sequence as herein described. In alternative embodiments, the composition comprises a pharmaceutically acceptable excipient and a DNA vector according to the present invention.

The composition may also include an adjuvant, or be administered either concomitantly with or sequentially with an adjuvant or immuno-stimulatory agent.

Thus it is an embodiment of the invention that the vectors of the invention be utilised with immunostimulatory agent. Preferably the immunostimulatory agent is administered at the same time as the nucleic acid vector of the invention and in preferred embodiments are formulated together. Such immunostimulatory agents include, (but this list is by no means exhaustive and does not preclude other agents): synthetic imidazoquinolines such as imiquimod [S-26308, R-837], (Harrison, et al. ‘Reduction of recurrent HSV disease using imiquimod alone or combined with a glycoprotein vaccine’, Vaccine 19: 1820-1826, (2001)); and resiquimod [S-28463, R-848] (Vasilakos, et al. ‘Adjuvant activites of immune response modifier R-848: Comparison with CpG ODN’, Cellular immunology 204: 64-74 (2000).), Schiff bases of carbonyls and amines that are constitutively expressed on antigen presenting cell and T-cell surfaces, such as tucaresol (Rhodes, J. et al. ‘Therapeutic potentiation of the immune system by costimulatory Schiff-base-forming drugs’, Nature 377: 71-75 (1995)), cytokine, chemokine and co-stimulatory molecules as either protein or peptide, this would include pro-inflammatory cytokines such as Interferons, particular interferons and GM-CSF, IL-1 alpha, IL-1 beta, TGF-alpha and TGF-beta, Th1 inducers such as interferon gamma, IL-2, IL-12, IL-15, IL-18 and IL-21, Th2 inducers such as IL-4, IL-5, IL-6, IL-10 and IL-13 and other chemokine and co-stimulatory genes such as MCP-1, MIP-1 alpha, MIP-1 beta, RANTES, TCA-3, CD80, CD86 and CD40L, other immunostimulatory targeting ligands such as CTLA-4 and L-selectin, apoptosis stimulating proteins and peptides such as Fas, (49), synthetic lipid based adjuvants, such as vaxfectin, (Reyes et al., ‘Vaxfectin enhances antigen specific antibody titres and maintains Th1 type immune responses to plasmid DNA immunization’, Vaccine 19: 3778-3786) squalene, alpha-tocopherol, polysorbate 80, DOPC and cholesterol, endotoxin, [LPS], Beutler, B., ‘Endotoxin, ‘Toll-like receptor 4, and the afferent limb of innate immunity’, Current Opinion in Microbiology 3: 23-30 (2000)); CpG oligo- and di-nucleotides, Sato, Y. et al., ‘Immunostimulatory DNA sequences necessary for effective intradermal gene immunization’, Science 273 (5273): 352-354 (1996). Hemmi, H. et al., ‘A Toll-like receptor recognizes bacterial DNA’, Nature 408: 740-745, (2000) and other potential ligands that trigger Toll receptors to produce Th1-inducing cytokines, such as synthetic Mycobacterial lipoproteins, Mycobacterial protein p19, peptidoglycan, teichoic acid and lipid A. Other bacterial derived immunostimulating proteins include, Cholera Toxin, E. Coli Toxin and mutant toxoids thereof.

Certain preferred adjuvants for eliciting a predominantly Th1-type response include, for example, a Lipid A derivative such as monophosphoryl lipid A, or preferably 3-de-O-acylated monophosphoryl lipid A. MPL® adjuvants are available from Corixa Corporation (Seattle, Wash.; see, for example, U.S. Pat. Nos. 4,436,727; 4,877,611; 4,866,034 and 4,912,094). CpG-containing oligonucleotides (in which the CpG dinucleotide is unmethylated) also induce a predominantly Th1 response. Such oligonucleotides are well known and are described, for example, in WO 96/02555, WO 99/33488 and U.S. Pat. Nos. 6,008,200 and 5,856,462. Immunostimulatory DNA sequences are also described, for example, by Sato et al., Science 273:352, 1996. Another preferred adjuvant comprises a saponin, such as Quil A, or derivatives thereof, including QS21 and QS7 (Aquila Biopharmaceuticals Inc., Framingham, Mass.); Escin; Digitonin; or Gypsophila or Chenopodium quinoa saponins.

Also provided are the use of a polynucleotide or protein according to the invention, or of a vector according to the invention, in the treatment or prophylaxis of MUC-1 expressing tumour, or metastases.

The present invention also provides methods of treating or preventing MUC-1 expressing tumours, any symptoms or diseases associated therewith, including metastases comprising administering an effective amount of a polynucleotide, a vector or a pharmaceutical composition according to the invention. Administration of a pharmaceutical composition may take the form of one or more individual doses, for example in a “prime-boost” therapeutic vaccination regime. In certain cases the “prime” vaccination may be via particle mediated DNA delivery of a polynucleotide according to the present invention, preferably incorporated into a plasmid-derived vector and the “boost” by administration of a recombinant viral vector comprising the same polynucleotide sequence, or boosting with the protein in adjuvant. Conversly the priming may be with the viral vector or with a protein formulation typically a protein formulated in adjuvant and the boost a DNA vaccine of the present invention.

As discussed above, the present invention includes expression vectors that comprise the nucleotide sequences of the invention. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for protein expression. Other suitable vectors would be apparent to persons skilled in the art. By way of further example in this regard we refer to Sambrook et al. Molecular Cloning: a Laboratory Manual. 2^{nd} Edition. CSH Laboratory Press. (1989).

Preferably, a polynucleotide of the invention, or for use in the invention in a vector, is operably linked to a control sequence which is capable of providing for the expression of the coding sequence by the host cell, i.e. the vector is an expression vector. The term “operably linked” refers to a juxtaposition wherein the components described are in a relationship permitting them to function in their intended manner. A regulatory sequence, such as a promoter, “operably linked” to a coding sequence is positioned in such a way that expression of the coding sequence is achieved under conditions compatible with the regulatory sequence.

The vectors may be, for example, plasmids, artificial chromosomes (e.g. BAC, PAC, YAC), virus or phage vectors provided with an origin of replication, optionally a promoter for the expression of the polynucleotide and optionally a regulator of the promoter. The vectors may contain one or more selectable marker genes, for example an ampicillin or kanamycin resistance gene in the case of a bacterial plasmid or a resistance gene for a fungal vector. Vectors may be used in vitro, for example for the production of DNA or RNA or used to transfect or transform a host cell, for example, a mammalian host cell e.g. for the production of protein encoded by the vector. The vectors may also be adapted to be used in vivo, for example in a method of DNA vaccination or of gene therapy.

Promoters and other expression regulation signals may be selected to be compatible with the host cell for which expression is designed. For example, mammalian promoters include the metallothionein promoter, which can be induced in response to heavy metals such as cadmium, and the β-actin promoter. Viral promoters such as the SV40 large T antigen promoter, human cytomegalovirus (CMV) immediate early (IE) promoter, rous sarcoma virus LTR promoter, adenovirus promoter, or a HPV promoter, particularly the HPV upstream regulatory region (URR) may also be used. All these promoters are well described and readily available in the art.

A preferred promoter element is the CMV immediate early promoter devoid of intron A, but including exon 1. Accordingly there is provided a vector comprising a polynucleotide of the invention under the control of HCMV IE early promoter.

Examples of suitable viral vectors include herpes simplex viral vectors, vaccinia or alpha-virus vectors and retroviruses, including lentiviruses, adenoviruses and adeno-associated viruses. Gene transfer techniques using these viruses are known to those skilled in the art. Retrovirus vectors for example may be used to stably integrate the polynucleotide of the invention into the host genome, although such recombination is not preferred. Replication-defective adenovirus vectors by contrast remain episomal and therefore allow transient expression. Vectors capable of driving expression in insect cells (for example baculovirus vectors), in human cells or in bacteria may be employed in order to produce quantities of the HIV protein encoded by the polynucleotides of the present invention, for example for use as subunit vaccines or in immunoassays. The polynucleotides of the invention have particular utility in viral vaccines as previous attempts to generate full-length vaccinia constructs have been unsuccessful.

The polynucleotides according to the invention have utility in the production by expression of the encoded proteins, which expression may take place in vitro, in vivo or ex vivo. The nucleotides may therefore be involved in recombinant protein synthesis, for example to increase yields, or indeed may find use as therapeutic agents in their own right, utilised in DNA vaccination techniques. Where the polynucleotides of the present invention are used in the production of the encoded proteins in vitro or ex vivo, cells, for example in cell culture, will be modified to include the polynucleotide to be expressed. Such cells include transient, or preferably stable mammalian cell lines. Particular examples of cells which may be modified by insertion of vectors encoding for a polypeptide according to the invention include mammalian HEK293T, CHO, HeLa, 293 and COS cells. Preferably the cell line selected will be one which is not only stable, but also allows for mature glycosylation and cell surface expression of a polypeptide. Expression may be achieved in transformed oocytes. A polypeptide may be expressed from a polynucleotide of the present invention, in cells of a transgenic non-human animal, preferably a mouse. A transgenic non-human animal expressing a polypeptide from a polynucleotide of the invention is included within the scope of the invention.

The invention further provides a method of vaccinating a mammalian subject which comprises administering thereto an effective amount of such a vaccine or vaccine composition. Most preferably, expression vectors for use in DNA vaccines, vaccine compositions and immunotherapeutics will be plasmid vectors.

DNA vaccines may be administered in the form of “naked DNA”, for example in a liquid formulation administered using a syringe or high pressure jet, or DNA formulated with liposomes or an irritant transfection enhancer, or by particle mediated DNA delivery (PMDD). All of these delivery systems are well known in the art. The vector may be introduced to a mammal for example by means of a viral vector delivery system.

The compositions of the present invention can be delivered by a number of routes such as intramuscularly, subcutaneously, intraperitonally or intravenously.

In a preferred embodiment, the composition is delivered intradermally. In particular, the composition is delivered by means of a gene gun (particularly particle bombardment) administration techniques which involve coating the vector on to a bead (eg gold) which are then administered under high pressure into the epidermis; such as, for example, as described in Haynes et al, J Biotechnology 44: 37-42 (1996).

In one illustrative example, gas-driven particle acceleration can be achieved with devices such as those manufactured by Powderject Pharmaceuticals PLC (Oxford, UK) and Powderject Vaccines Inc. (Madison, Wis.), some examples of which are described in U.S. Pat. Nos. 5,846,796; 6,010,478; 5,865,796; 5,584,807; and EP Patent No. 0500 799. This approach offers a needle-free delivery approach wherein a dry powder formulation of microscopic particles, such as polynucleotide, are accelerated to high speed within a helium gas jet generated by a hand held device, propelling the particles into a target tissue of interest, typically the skin. The particles are preferably gold beads of a 0.4-4.0 μm, more preferably 0.6-2.0 μm diameter and the DNA conjugate coated onto these and then encased in a cartridge or cassette for placing into the “gene gun”.

In a related embodiment, other devices and methods that may be useful for gas-driven needle-less injection of compositions of the present invention include those provided by Bioject, Inc. (Portland, Oreg.), some examples of which are described in U.S. Pat. Nos. 4,790,824; 5,064,413; 5,312,335; 5,383,851; 5,399,163; 5,520,639 and 5,993,412.

The vectors which comprise the nucleotide sequences encoding antigenic peptides are administered in such amount as will be prophylactically or therapeutically effective. The quantity to be administered, is generally in the range of one picogram to 1 milligram, preferably 1 picogram to 10 micrograms for particle-mediated delivery, and 10 micrograms to 1 milligram for other routes of nucleotide per dose. The exact quantity may vary considerably depending on the weight of the patient being immunised and the route of administration.

It is possible for the immunogen component comprising the nucleotide sequence encoding the antigenic peptide, to be administered on a once off basis or to be administered repeatedly, for example, between 1 and 7 times, preferably between 1 and 4 times, at intervals between about 1 day and about 18 months. Once again, however, this treatment regime will be significantly varied depending upon the size of the patient, the disease which is being treated/protected against, the amount of nucleotide sequence administered, the route of administration, and other factors which would be apparent to a skilled medical practitioner. The patient may receive one or more other anti cancer drugs as part of their overall treatment regime.

Suitable techniques for introducing the naked polynucleotide or vector into a patient also include topical application with an appropriate vehicle. The nucleic acid may be administered topically to the skin, or to mucosal surfaces for example by intranasal, oral, intravaginal or intrarectal administration. The naked polynucleotide or vector may be present together with a pharmaceutically acceptable excipient, such as phosphate buffered saline (PBS). DNA uptake may be further facilitated by use of facilitating agents such as bupivacaine, either separately or included in the DNA formulation. Other methods of administering the nucleic acid directly to a recipient include ultrasound, electrical stimulation, electroporation and microseeding which is described in U.S. Pat. No. 5,697,901.

Uptake of nucleic acid constructs may be enhanced by several known transfection techniques, for example those including the use of transfection agents. Examples of these agents includes cationic agents, for example, calcium phosphate and DEAE-Dextran and lipofectants, for example, lipofectam and transfectam. The dosage of the nucleic acid to be administered can be altered.

A nucleic acid sequence of the present invention may also be administered by means of transformed cells. Such cells include cells harvested from a subject. The naked polynucleotide or vector of the present invention can be introduced into such cells in vitro and the transformed cells can later be returned to the subject. The polynucleotide of the invention may integrate into nucleic acid already present in a cell by homologous recombination events. A transformed cell may, if desired, be grown up in vitro and one or more of the resultant cells may be used in the present invention. Cells can be provided at an appropriate site in a patient by known surgical or microsurgical techniques (e.g. grafting, micro-injection, etc.)

### EXAMPLES

1.1 Generation of Constructs

A schematic of the relationship between all the constructs detailed below can be found in FIG. 1.

1.2 Construction the Full-Length MUC-1 Expression Cassette

The starting point for the construction of a MUC-1 expression cassette was the plasmid pcDNA3-FL-MUC-1 (ICRF, London). This plasmid has a pcDNA3 backbone (Invitrogen) containing a full-length MUC-1 (FL-MUC1) cDNA cassette cloned at the BamHI site. On the basis of restriction mapping performed at the ICRF, the MUC-1 gene has approximately 32 VNTR units (variable number of tandem repeats). The presence of MUC-1 was confirmed by fluorescent sequencing using the primers 2004MUC1-2014MUC1 (Appendix A). The MUC-1 sequence on which the FL-MUC1 sequence is based is shown in FIG. 2. In the first stage of the cloning process, a BamHI fragment containing the full-length MUC-1 cDNA sequence was isolated and cloned into the BamHI site of the expression vector pcDNA3.I(+)/Hygro (Invitrogen), generating plasmid JNW278. The correct orientation of the fragment, relative to the CMV promoter, was confirmed by PCR and fluorescent sequencing.

The next stage of cloning involved the removal of the 3′ untranslated region (UTRs) and replacement with improved restriction enzyme sites to facilitate future cloning procedures. A fragment of MUC-1 was PCR amplified with primers 2062MUC1 and 2063MUC1 (Appendix A) using JNW278 as a template and restricted using BstXI and XhoI. In parallel, plasmid JNW278 was restricted using BstXI and XhoI. The purified vector backbone was ligated with the PCR fragment generating plasmid JNW314. Restriction analysis and fluorescent sequencing confirmed the presence of the correct fragment.

In parallel, the 5′ UTR was removed and replaced with an optimal Kozak sequence and improved restriction enzyme sites. JNW278 was restricted with NheI-XhoI removing the entire MUC-1 cDNA sequence. A fragment of MUC-1 was PCR amplified with PCR primers 2060MUC1 and 2061MUC1 (Appendix A), restricted NheI and XhoI, and ligated into the vector backbone generating plasmid JNW294.

In the next stage of the cloning JNW294 was restricted using BsaMI, releasing two fragments (of approx. 2.3 kbp and 3.2 kbp). The larger of these two fragments (Fragment A) was isolated and purified. In parallel, JNW314 was restricted with BsaMI and the larger of the two fragments (Fragment B, approx. 7 kbp in size) was isolated and purified. Fragment A and B were ligated together generating plasmid JNW340. The correct orientation was confirmed by restriction mapping using Nhe-XhoI and separately, XbaI.

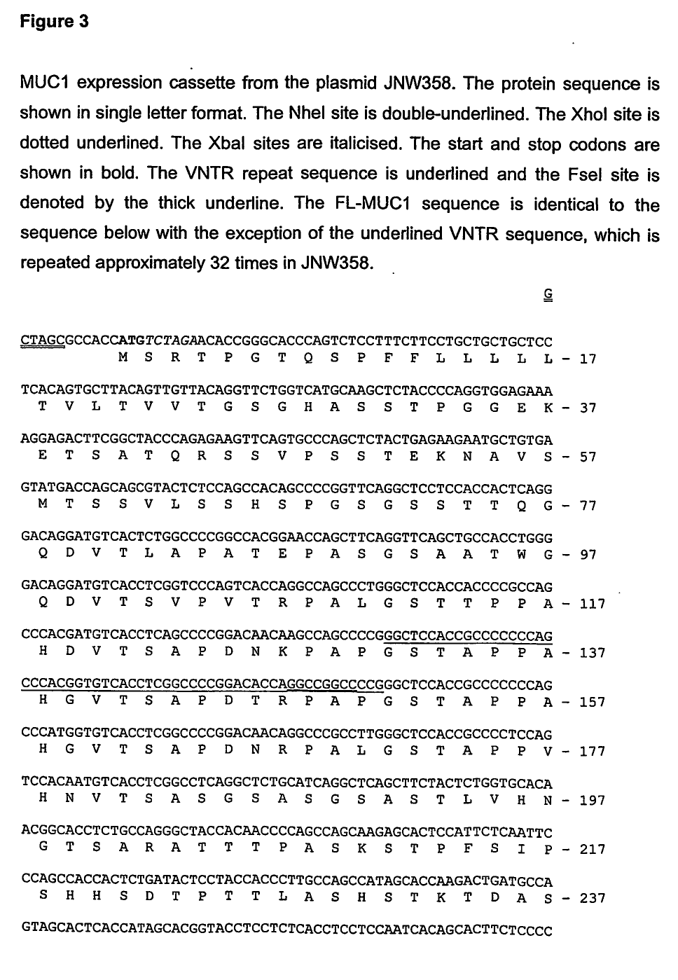

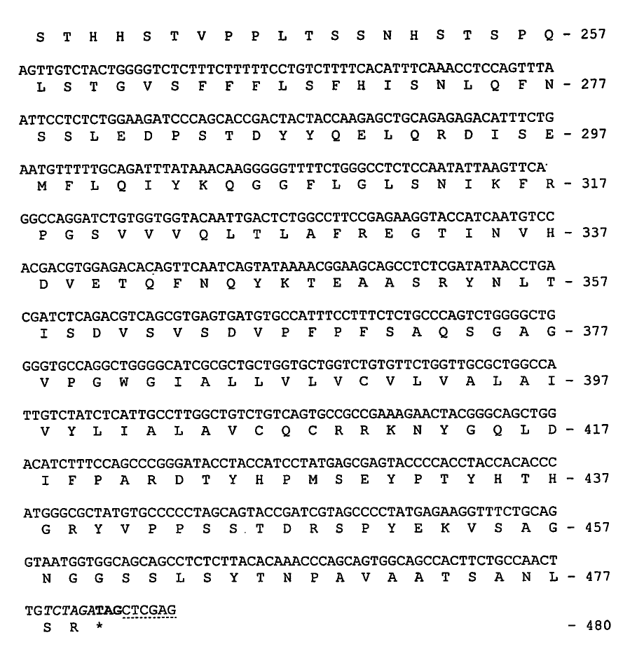

In the final stage of the cloning, an expression cassette was isolated from JNW340 by restriction digest with NheI and XhoI, releasing a fragment of approximately 4 kbp. The expression plasmid pVAC1 (Thomsen Immunology 95: 51OP106, 1998) was restricted with NheI-XhoI and ligated with the MUC-1 cassette, generating the full-length MUC-1 expression plasmid JNW358. The correct orientation of the MUC-1 sequence relative to the CMV promoter was confirmed by restriction digest and fluorescent sequencing. The sequence of the MUC-1 expression cassette is shown in FIG. 3.

1.3 Construction of a MUC-1 Vector Containing One VNTR Unit

The starting point for construction of a MUC-1 expression cassette containing one VNTR units is JNW278. A unique feature of the highly repetitive VNTR DNA sequence is the presence of an FseI restriction site in each of the repeat units. JNW278 was restriction digested to completeness using FseI, the vector backbone isolated and religated to generate plasmid JNW283. The presence of a single VNTR unit was confirmed by restriction analysis, PCR and by fluorescent sequencing. The MUC-1 sequence of JNW283 is shown in FIG. 2.

1.4 Construction of a MUC-1 Expression Vector Containing One VNTR Unit

To transfer the MUC-1 cassette containing one VNTR unit from JNW283 into the expression plasmid pVAC, the following cloning steps were taken. The first stage of cloning involved the removal of the 5′ and 3′ untranslated region (UTRs) and replacement with improved restriction enzyme sites to facilitate future cloning procedures. A fragment of MUC-1 was PCR amplified with primers 2060MUC1 and 2062MUC1 using JNW283 as a template and restricted using NheI and XhoI. In parallel, plasmid pVAC was restricted using NheI and XhoI. The purified vector backbone was ligated with the PCR fragment generating plasmid JNW322. Restriction analysis and fluorescent sequencing confirmed the presence of the correct fragment.

1.5 Construction of a MUC-1 Cassette Containing a Small Number of VNTR Units

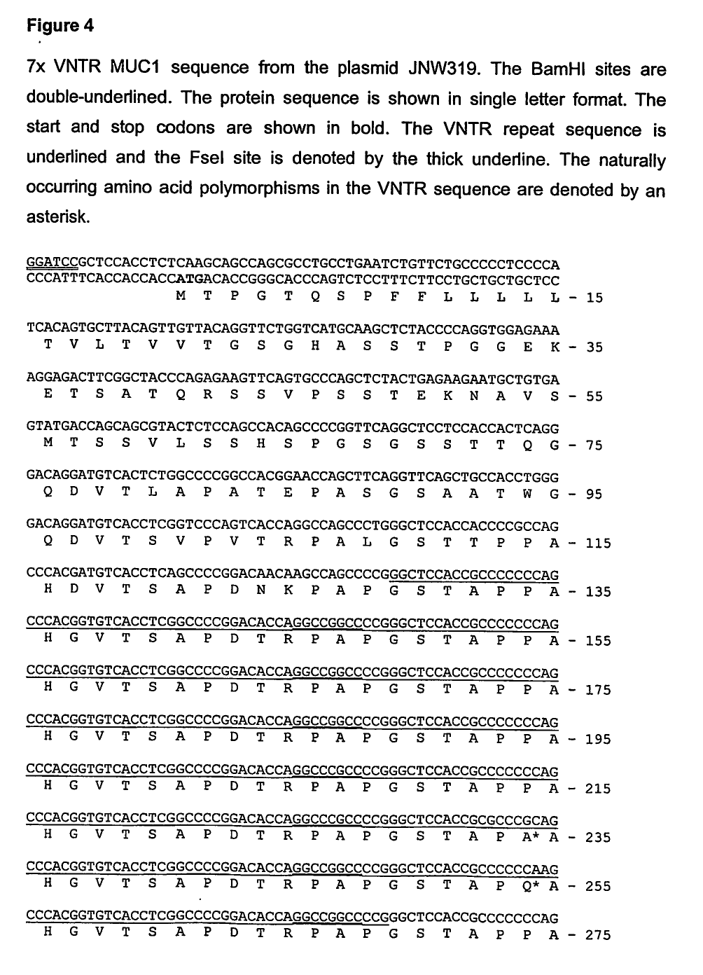

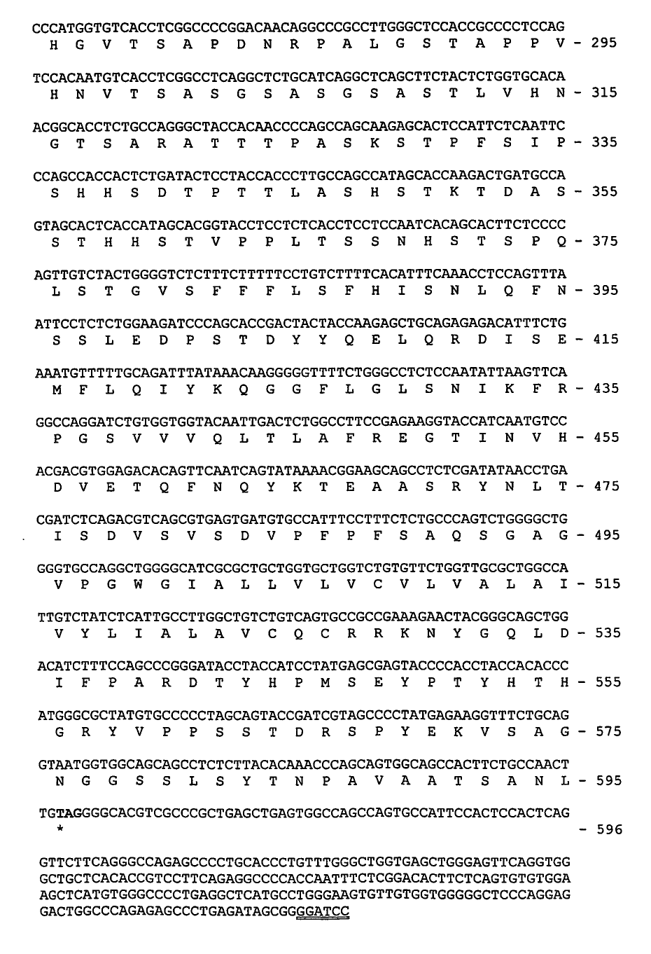

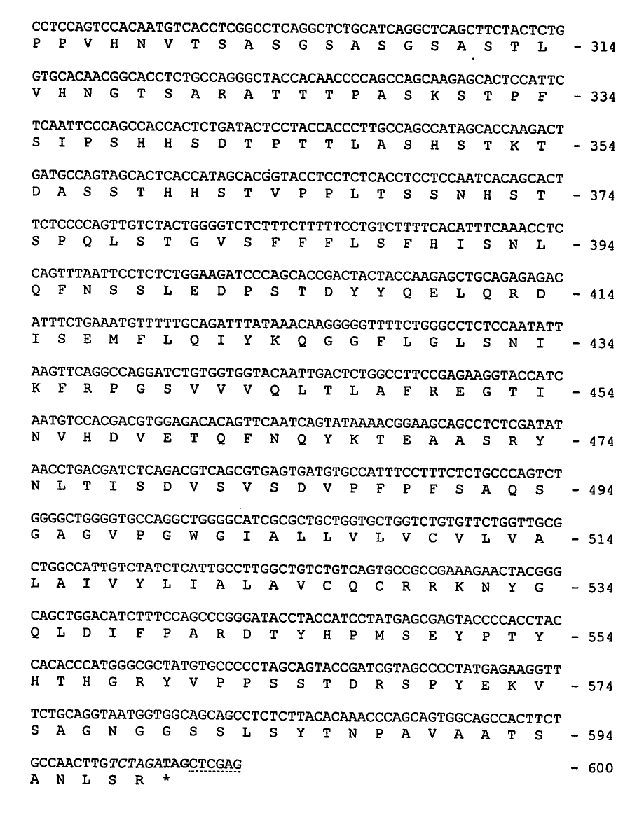

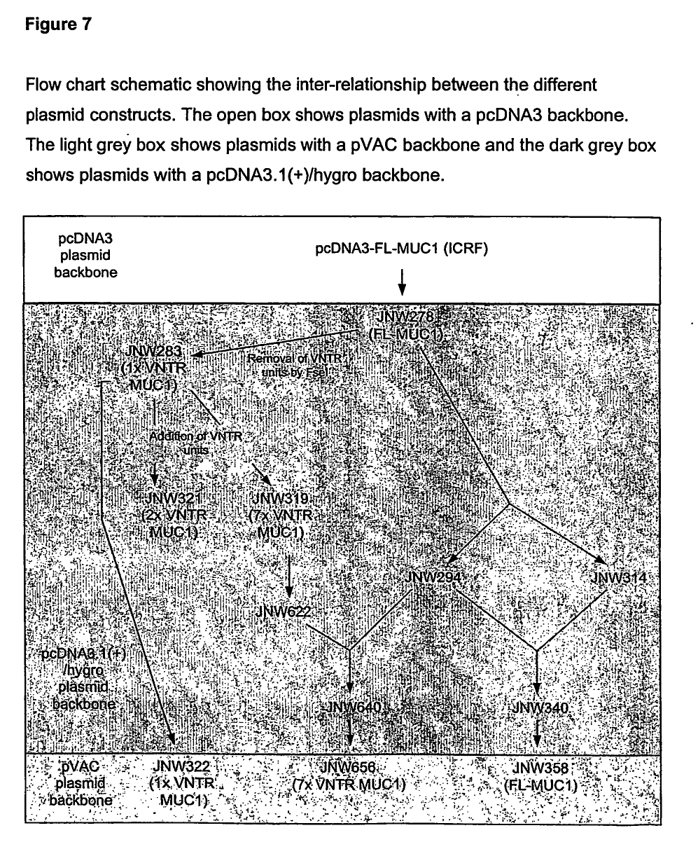

The starting point for the construction of a MUC-1 expression cassette containing a small number of VNTR units is JNW283 which was linearised using FseI. The VNTR units were generated by partial digest of plasmid JNW278 with FseI to release a ladder of short fragments varying in size from 60 bp—equivalent to one VNTR unit—to approximately 420 bp which corresponds to seven VNTR units. The ladder of VNTR fragments generated by a partial digest of JNW278 is shown in FIG. 7. The fragments of 60-500 bp were purified by gel extraction and ligated with FseI-linearised JNW283. Clones were initially screened by PCR using the primers 2005MUC1 and 2013MUC1 which are positioned 5′ and 3′ respectively of the VNTR region of MUC-1. The PCR was set up in such a way that clones which containing multiple VNTR units would yield a PCR fragment larger than the PCR fragment corresponding to the one VNTR unit of JNW283. PCR positive clones were subject to further analysis by restriction digest and fluorescent sequencing to confirm the number of VNTR units present. Using this protocol, a number of different plasmids were obtained including JNW319 which possesses seven VNTR units in total and JNW321 which possesses two VNTR units. The sequences of JNW319 and JNW321 are shown in FIGS. 4 & 5. The VNTR units of JNW319 show polymorphisms that are present in the wider population (denoted by the asterisks).

1.6 Construction of a MUC-1 Expression Vector Containing Seven VNTR Units

To transfer the MUC-1 cassette containing seven VNTR units into the expression plasmid pVAC, the following cloning steps were taken. The first stage of cloning involved the removal of the 3′ untranslated region (UTRs) and replacement with improved restriction enzyme sites to facilitate future cloning procedures. A fragment of MUC-1 was PCR amplified with primers 2062MUC1 and 2063MUC1 using JNW278 as a template and restricted using BstXI and XhoI. In parallel, plasmid JNW319 was restricted using BstXI and XhoI. The purified vector backbone was ligated with the PCR fragment generating plasmid JNW622. Restriction analysis and fluorescent sequencing confirmed the presence of the correct fragment.

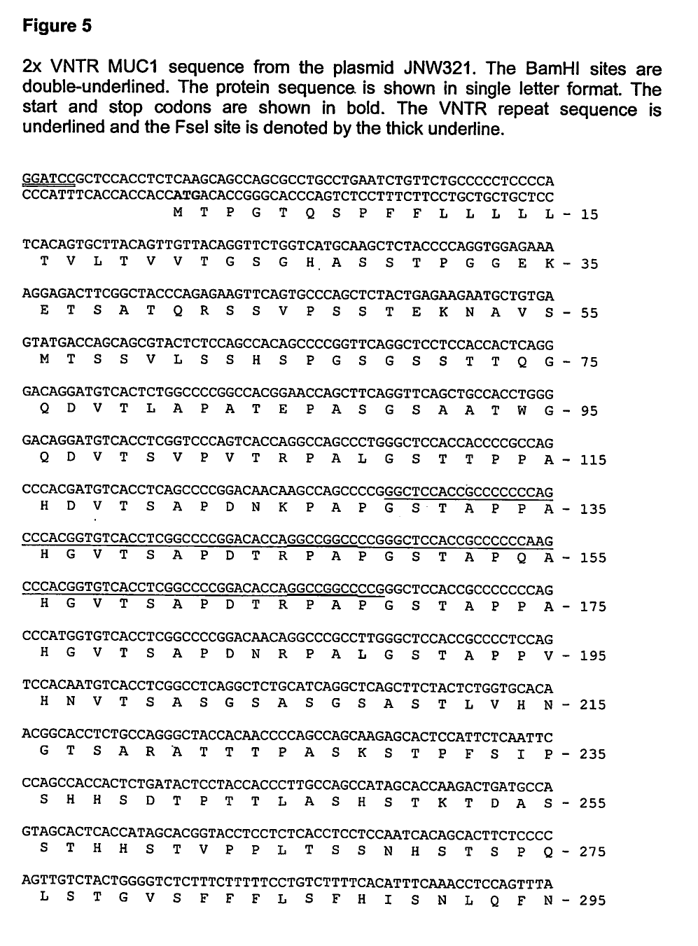

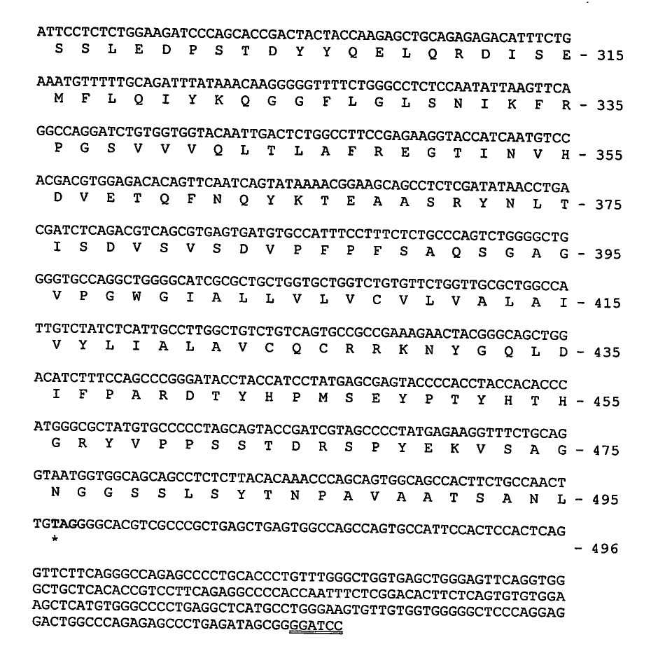

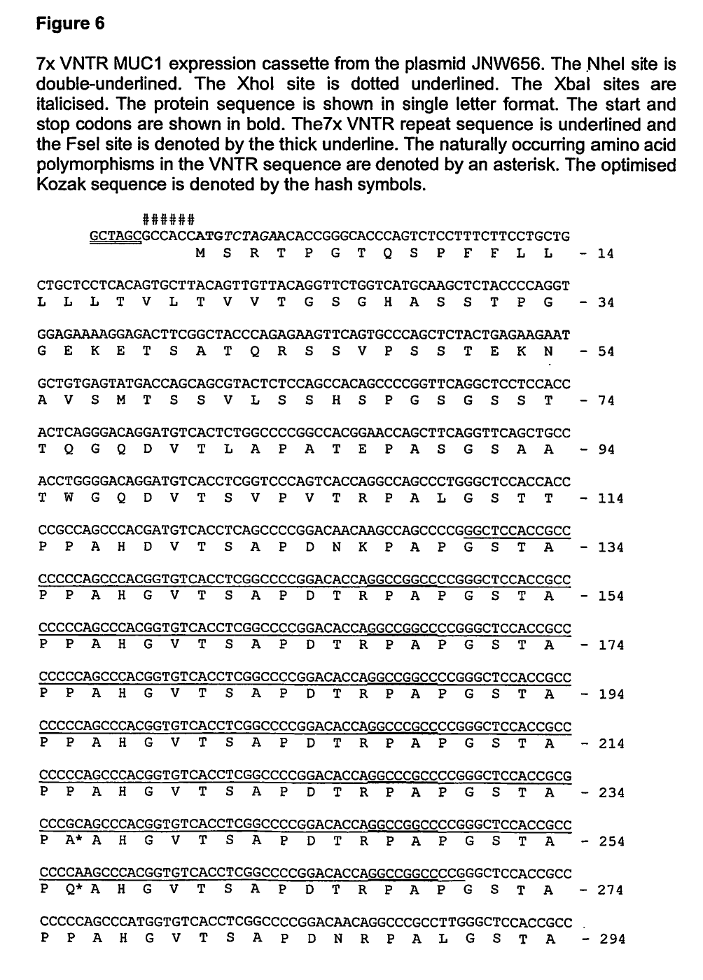

In the next stage of the cloning JNW294 was restricted using BsaMI, releasing two fragments (of approx. 2.3 kbp and 3.2 kbp). The larger of these two fragments (Fragment A) was isolated and purified. In parallel, JNW622 was restricted with BsaMI and the larger of the two fragments Fragment C, approx. 4 kbp in size) was isolated and purified. Fragment A and C were ligated together generating plasmid JNW640. The correct orientation was confirmed by restriction mapping using XbaI and fluorescent sequencing. In the final stage of cloning, the MUC-1 cassette from JNW640 was isolated following restriction with NheI and XhoI and ligated with pVAC (also linearised with NheI and XhoI), generating plasmid JNW656. The sequence of the MUC-1 expression cassette was confirmed by fluorescent sequencing and is shown in FIG. 6.

1.7 Purification of VNTR Units

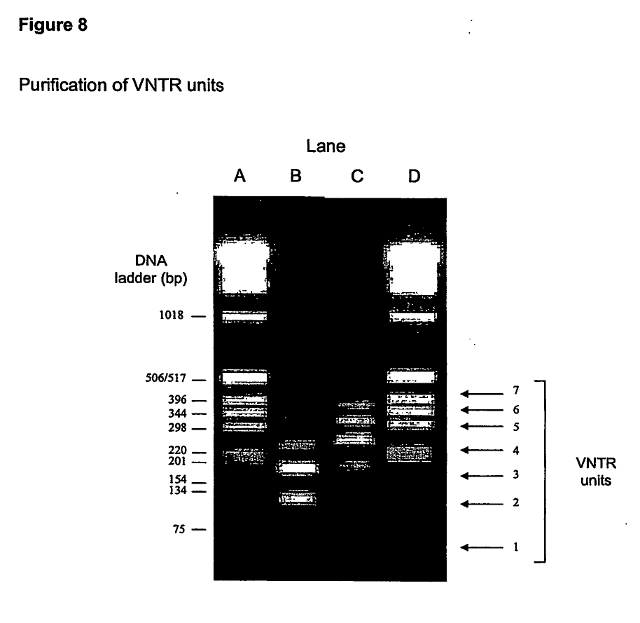

Following digestion of JNW278 (FL-MUC1) with FseI, a ladder of VNTR was released, ranging from 60 bp (equivalent to one VNTR unit) to 420 bp (equivalent to seven VNTR units). Following electrophoresis, the fragments were isolated from the agarose gel and purified. FIG. 8 shows two ladders of VNTR units. The DNA markers are shown in Lanes A & D. Lane B shows the ladder representing VNTR units in the range 60-240 bp. Lane C shows the ladder representing VNTR units in the range 180-420 bp. These fragments were subsequently ligated into the FseI-linearised JNW283 to build up a MUC-1 gene containing 2, and 7 VNTR units. Other constructs containing 3, 4,5 or 6 VNTR units can be made in an analogous fashion (See FIG. 7).

2: Preparation of Constructs for Cutaneous Gene Gun Immunisation

Plasmid DNA was precipitated onto 2 μm diameter gold beads using calcium chloride and spermidine. Loaded beads were coated onto Tefzel tubing as described (Eisenbraum et al, 1993; Pertmer et al, 1996). Particle bombardment was performed using the Accell gene delivery system (PCT WO 95/19799). For each plasmid, female C56B1/6 mice were immunised with 3 administrations of plasmid on days 0, 21 and 42. Each administration consisted of two bombardments with DNA/gold, providing a total dose of approximately 4-5 μg of plasmid.

2.1 Intramuscular (i.m.) DNA Immunization

Female C57B1/6 Mice were immunised intramuscularly into the hind leg with 50 μg of DNA in PB S on day 0, 21 and 42.

2.2 Tumour Cell Injection

Two sets of tumour regression experiment were performed in which, in the first experiment 0.5×10⁶ tumour cells were subcutaneously injected in the right flank of anaesthetized mice two weeks after the last immunisation. In the second experiment a much more aggressive model was used in which the animal received 1.0×10⁶ tumour cells. Tumour growth was monitored twice a week using vernier calipers in two dimensions. Tumour volumes were calculated as (a×b²)/2, where a represents the largest diameter and b the smallest diameter. The experimental endpoint (death) was defined as the time point at which tumour diameter reached 15 mm.

3: Testing of Constructs

3.1.1 Materials & Methods

B16F0 and B16F0-MUC1 Tumour Cells

B16F0 (murine metastatic melanoma) transfected with an expression vector for the human cDNA MUC-1 were obtained from Glaxo Wellcome U.S. Cells were cultivated as adherent monolayers in DMEM supplemented with 10% heat inactivated fetal calf serum, 2 mM L-glutamine, 100 U/ml penicillin, 100 μg/ml streptomycin and 1 mg/ml of neomycin antibiotic (G148). For use in ELISPOT assays cells were removed from flasks using Versene and irradiated (16,000 Rads).

3.1.2 Construction of EL4 tumour cells expressing MUC-1

EL4 cells were cultured in RPMI complete media containing 10% FCS, 100 U/ml penicillin, 100 μg/ml streptomycin, 2 mM L-glutamine, 50 μM 2-mercaptoethanol. JNW278 (full-length MUC-1) was linearised with FspI, purified by phenol:chloroform:iso-amyl alcohol (25:24:1) extraction followed by ethanol precipitation. 2×10⁷ cells in 0.5 ml RPMI complete media were mixed with 20μg of linearised DNA in a 0.4 mm BIORAD cuvette. The cells were transfected by electroporation at 320V, 960 μF. After electroporation, the cell suspension was transferred to 30 ml of pre-warmed RPMI complete media and incubated for 24 hours to allow recovery. The cells were placed under selection in RPMI complete media containing 500 μg/ml hygromycin and incubated for 7-10 days. Surviving cells were diluted into 96-well U-bottomed plates at 0.5 cells/well in 200 μl of RPMI complete media including 500 μg/ml hygromycin. 8-10 days later, clones were transferred into 24-well plates. At this stage, the MUC-1 expression profile was assessed by flow cytometry and positive, uniform clones were maintained for further analysis.

3.2 Elispot Assays for T Cell Responses to the MUC-1 Gene Product

3.2.1 Preparation of Splenocytes

Spleens were obtained from immunised animals at 7 days post boost, which was either at day 28 or day 49. Spleens were processed by grinding between glass slides to produce a cell suspension. Red blood cells were lysed by ammonium chloride treatment and debris was removed to leave a fine suspension of splenocytes. Cells were resuspended at a concentration of 8×10⁶/ml in RPMI complete media for use in ELISPOT assays.

3.3 Screening of Peptide Library

A peptide library covering the entire sequence of MUC-1 was purchased from Mimotopes. The library contained 116 15 mer peptides overlapping by 11 amino acids peptides covering the entire sequence of MUC-1 (including 1 copy of the tandem repeat region). Peptides are represented by numbers from 184-299. For screening of the peptide library, peptides were used at a final concentration of 10 μM in IFNγ and IL-2 ELISPOTS using the protocol described below. For IFNγ ELISPOTS, Il-2 was added to the assays at 10 ng/ml. Splenocytes used for the screening were taken at day 49 from C57BL/6 mice or CBA mice immunised with FL MUC1 at Days 0, 21 and 42.

3.4 Epitope Mapping Two MUC-1 regions showing good reactivity in C57BL/6 mice were selected for further study. These were regions covered by peptides 222-225 and 238-239. By flow cytometry (protocol below) it was shown that the cells producing IFNγ in response to these peptides were CD8 cells. To map the epitopes further 8 and 9 mer peptides overlapping by 7 or 8 amino acids respectively were ordered from Mimotopes. These were tested in IFNγ ELISPOT using splenocytes from animals immunised as detailed above. Two immunodominant peptides were identified, SAPDNRPAL and PTTLASHS.

3.5 ELISPOT Assay

Plates were coated with 15 μg/ml (in PBS) rat anti mouse IFNγ or rat anti mouse IL-2 (Pharmingen). Plates were coated overnight at +4° C. Before use the plates were washed three times with PBS. Splenocytes were added to the plates at 4×10⁵ cells/well. Peptide SAPDNRPAL was used in assays at a final concentration of 10 nM. Peptide PAHGVTSAPDTRPAPGSTAPPAHGV (25 mer peptide) was used at a final concentration of 25 μM. These peptides were obtained from Genemed Synthesis. Peptides identified from library screening and epitope mapping studies were also used in ELISPOT assays: 203 (DVTLAPATEPATEPA) at 10 μM, 299 (LSYTNPAVAATSANL) at 10 μM, PTTLASHS at 1 μM (Mimotopes). Irradiated tumour cells B16, B16-MUC1 and EL4, EL4-278 were used at a tumour cell: effector ratio of 1:4. ELISPOT assays were carried out in the presence of either IL-2 (10 ng/ml), IL-7 (10 ng/ml) or no cytokine. Total volume in each well was 200 μl. Plates containing peptide stimulated cells were incubated for 16 hours in a humidified 37° C. incubator while those containing tumour cells as stimulators were incubated for 40 hours.

3.5.1 Development of ELISPOT Assay Plates.

Cells were removed from the plates by washing once with water (with 1 minute soak to ensure lysis of cells) and three times with PBS. Biotin conjugated rat anti mouse IFNγ or IL-2 (Phamingen) was added at 1 μg/ml in PBS. Plates were incubated with shaking for 2 hours at room temperature. Plates were then washed three times with PBS before addition of Streptavidin alkaline phosphatase (Caltag) at 1/1000 dilution. Following three washes in PBS spots were revealed by incubation with BCICP substrate (Biorad) for 1545 mins. Substrate was washed off using water and plates were allowed to dry. Spots were enumerated using an image analysis system devised by Brian Hayes, Asthma Cell Biology unit, GSK.

3.6 Flow Cytometry to Detect IFNγ Production from T Cells in Response to Peptide Stimulation.

Splenocytes were resuspended at 4×10⁶/ml. Peptide was added at a final concentration of 10 μM and IL-2 at a final concentration of 10 ng/ml. Cells were incubated at 37° C. for 3 hours, Brefeldin A was added at 10 μg/ml, and incubation continued overnight. Cells were washed with FACS buffer (PBS+2.5% FCS+0.1% azide) and stained with anti CD4 Cychrome and anti CD8 FITC (Pharmingen). Cells were washed and fixed with Medium A from Caltag Fix and Perm kit for 15 mins followed by washing and addition of anti IFNγ PE (Pharmingen) diluted in Medium B from the Fix and Perm kit. After 30 mins incubation cells were washed and analysed using a FACSCAN. A total of 500,000 cells were collected per sample and subsequently CD4 and CD8 cells were gated to determine the populations of cells secreting IFNγ in response to each peptide.

3.7 ELISA Assay for Antibodies to the MUC-1 Gene Product

Serum samples were obtained from the animals by venepuncture on days—1, 21, 49 and 56, and assayed for the presence of anti-MUC-1 antibodies. ELISA was performed using Nunc Maxisorp plates coated overnight at 4° C. with 3 μg/ml of wild type MUC-01 peptide sequence (40-mer corresponding to 2 tandem repeat sequence, PAHGVTSAPDTRPAPGSTAPPAHGVTSAPDTRPAPGSTAP). After washing with TBS-Tween (Tris-buffered saline, pH 7.4 containing 0.05% of Tween 20) the plates were blocked with 3% BSA in TBS-Tween buffer for 2 h at room temperature. All sera were incubated at 1:100 dilution for 1 h at RT in TBS-Tween buffer. Antibody binding was detected using HRP-conjugated rabbit anti-mouse immunoglobulins (#P0260, Dako) at 1:2000 dilution in TBS-Tween buffer. Plates were washed again and bound conjugate detected using Fast OPD colour reagents (Sigma, UK). The reaction was stopped by the addition of 3M sulphuric acid, and the OPD product quantitated by measuring the absorbance at 490 nm.

3.8 Flow Cytometric Analysis of Sera from Immunised Mice

In order to demonstrate that antibodies evoked by these vaccines are capable of recognising tumour cells, samples of antisera from PMID immunised mice were used to label various tumour cell lines, and the labeling visualised by flow cytometry. Cells (T47-D, MCF-7, EL4, EL4-278, B16F0 and B16F0MUC1;1×10⁶) were washed in PBS buffer supplemented with 5% FCS and incubated at 4° C. for 15 min with mouse sera at 1:100 dilution. After washing, cells were incubated with the second antibody (Sheep anti-mouse IgG, Dako, Denmark, at 1:50 dilution) under the same conditions. Control cells were incubated with FACS buffer instead of the first step antibody prior to staining with the second step reagent. FACS analysis was performed using a FACScan (Becton Dickinson). 1000-10000 cells per sample were simultaneously measured for FSC (forward angle light scatter) and SSC (integrated light scatter) as well as green (FL1) fluorescence (expressed as logarithm of the integrated fluorescence light). Recordings were made excluding aggregates whose FCS were out of range. Data were expressed as histograms plotted as number of cells (Y-axis) versus fluorescence intensity (X-axis) for the different types of mouse sera bound to the surface of the tumour cells.

3.9 Transient Transfection Assays

MUC-1 expression from various DNA constructs was analysed by transient transfection of the plasmids into CHO (Chinese hamster ovary) cells followed by either Western blotting on total cell protein, or by flow cytometric analysis of cell membrane expressed MUC-1. Transient transfections were performed with the Transfectam reagent (Promega) according to the manufacturer's guidelines. In brief, 24-well tissue culture plates were seeded with 5×10⁴ CHO cells per well in 1 ml DMEM complete medium (DMEM, 10% FCS, 2 mM L-glutamine, penicillin 100 IU/ml, streptomycin 100 μg/ml) and incubated for 16 hours at 37° C. 0.5 μg DNA was added to 25 μl of 0.3M NaCl (sufficient for one well) and 2 μl of Transfectam was added to 25 μl of Milli-Q. The DNA and Transfectam solutions were mixed gently and incubated at room temperature for 15 minutes. During this incubation step, the cells were washed once in PBS and covered with 150 μl of serum free medium (DMEM, 2 mM L-glutamine). The DNA-Transfectam solution was added drop wise to the cells, the plate gentle shaken and incubated at 37° C. for 4-6 hours. 500 μl of DMEM complete medium was added and the cells incubated for a further 48-72 hours at 37° C.

3.10 Flow Cytometric Analysis of CHO Cells Transiently Transfected with MUC-1 Plasmids

Following transient transfection, the CHO cells were washed once with PBS and treated with a Versene (1:5000)/0.025% trypsin solution to transfer the cells into suspension. Following trypsinisation, the CHO cells were pelleted and resuspended in FACS buffer (PBS, 4% FCS, 0.01% sodium azide). The primary antibody, ATR1 was added to a final concentration of 15 μg/ml and the samples incubated on ice for 15 minutes. Control cells were incubated with FACS buffer in the absence of ATR1. The cells were washed three times in FACS buffer, resuspended in 100 μl FACS buffer containing 10 μl of the secondary antibody goat anti-mouse immunoglobulins FITC conjugated F(ab′)₂ (Dako, F0479) and incubated on ice for 15 minutes. Following secondary antibody staining, the cells were washed three times in FACS buffer. FACS analysis was performed using a FACScan (Becton Dickinson). 1000-10000 cells per sample were simultaneously measured for FSC (forward angle light scatter) and SSC (integrated light scatter) as well as green (FL1) fluorescence (expressed as logarithm of the integrated fluorescence light). Recordings were made excluding aggregates whose FCS were out of range. Data were expressed as histograms plotted as number of cells (Y-axis) versus fluorescence intensity (X-axis).

3.11 Western Blot Analysis of CHO Cells Transiently Transfected with MUC-1 Plasmids

The transiently transfected CHO cells were washed with PBS and treated with a Versene (1:5000)/0.025% trypsin solution to transfer the cells into suspension. Following trypsinisation, the CHO cells were pelleted and resuspended in 50 μl of PBS. An equal volume of 2× TRIS-Glycine SDS sample buffer (Invitrogen) containing 50 mM DTT was added and the solution heated to 95° C. for 5 minutes. 1-20 μl of sample was loaded onto a 4-20% TRIS-Glycine Gel 1.5 mm (Invitrogen) and electrophoresed at constant voltage (125V) for 90 minutes in 1× TRIS-Glycine buffer (Invitrogen). A pre-stained broad range marker (New England Biolabs, #P7708S) was used to size the samples. Following electrophoresis, the samples were transferred to Immobilon-P PVDF membrane (Millipore), pre-wetted in methanol, using an Xcell III Blot Module (Invitrogen), 1× Transfer buffer (Invitrogen) containing 20% methanol and a constant voltage of 25V for 90 minutes. The membrane was blocked overnight at 4° C. in TBS-Tween (Tris-buffered saline, pH 7.4 containing 0.05% of Tween 20) containing 3% dried skimmed milk (Marvel). The primary antibody (ATR1) was diluted 1:100 and incubated with the membrane for 1 hour at room temperature. Following extensive washing in TBS-Tween, the secondary antibody was diluted 1:2000 in TBS-Tween containing 3% dried skimmed milk and incubated with the membrane for one hour at room temperature. Following extensive washing, the membrane was incubated with Supersignal West Pico Chemiluminescent substrate (Pierce) for 5 minutes. Excess liquid was removed and the membrane sealed between two sheets of cling film, and exposed to Hyperfilm ECL film (AmershamPharmaciaBiotech) for 1-30 minutes.

4. Results

4.1 Comparison of Gene Gun and Intramuscular Injection

The FL-MUC1 expression cassette in the plasmid pcDNA3-FL-MUC1, was administered to mice by PMID and by intramuscular injection.

4.2 Comparison of Antibody Responses

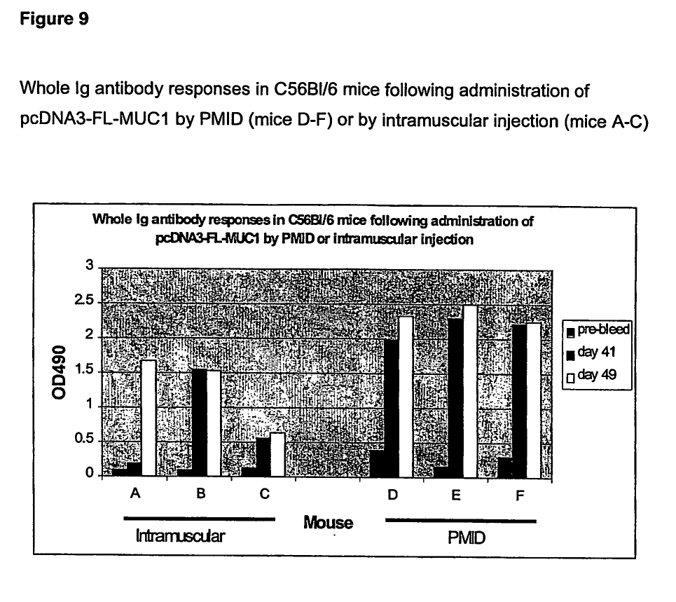

The anti-MUC1 antibody responses following immunisation by intramuscular injection (mouse A-C) and by PMID (mouse D-F) are shown in FIG. 9. The results show that the administration by PMID induces a more robust antibody response with faster kinetics, with 3 of 3 mice responding at day 41. In contrast, only one mouse immunised by the intramuscular route showed good antibody responses at day 41. Even after a further boost at day 42, only 2 of 3 mice showed levels of MUC-1 antibodies comparable to those of the PMID immunised mice.

4.3 Comparison of Cellular Responses

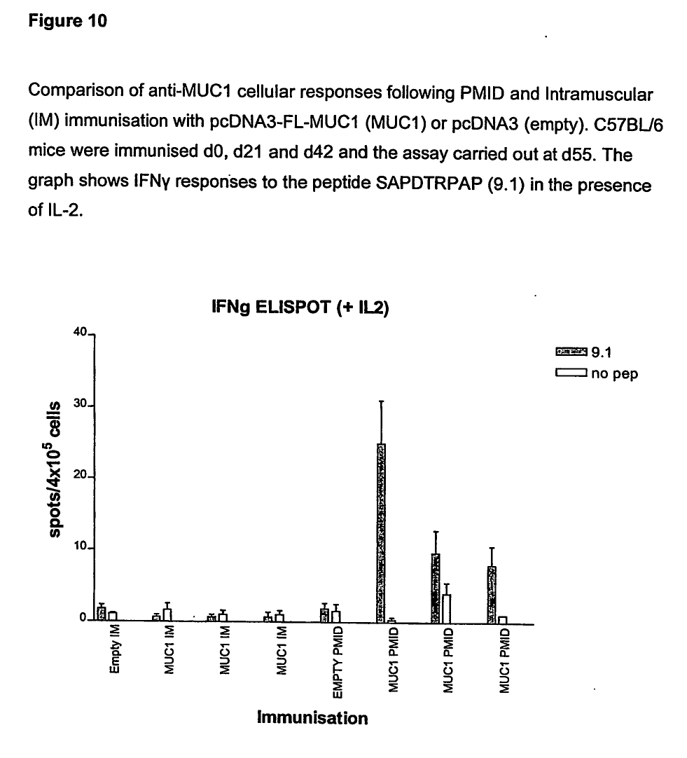

The cellular responses following PMID or Intramuscular (IM) immunisation with pcDNA3 (empty vector) or pcDNA3-FL-MUC1 were assessed by ELISPOT following primary immunisation at day 0 and two boosts at day 21 and day 42. The assay was carried out at day 13 post the 2^{nd} boost. Splenocytes were stimulated with the peptide SAPDTRPAP (9.1) that has previously been described in the literature as a good H-2 Kb epitope. The IFNγ responses, FIG. 10 shows that 100% of the mice immunised by PMID have detectable responses to the peptide whilst no responses were detected in the mice immunised intramuscularly.

4.4 In Vitro Expression of MUC-1 Constructs—Western Blot

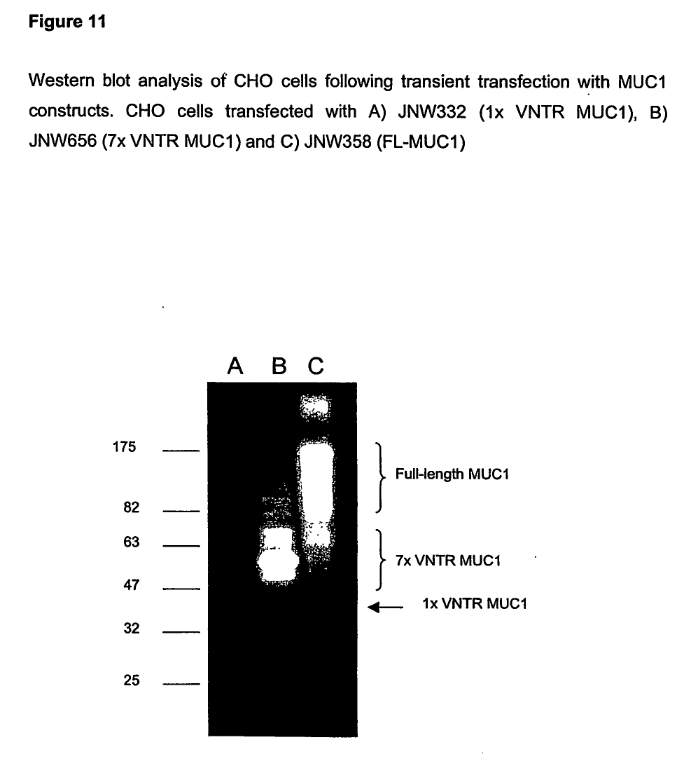

FIG. 11 shows the results of a Western blot of total cell protein for MUC-1 following transient transfection of various MUC-1 constructs into CHO cells. The data shows that the FL-MUC1 construct (JNW358) generates a smear at 83-175 kDa, consistent with the predicted molecular weight of 108 kDa and heterogeneous but extensive glycosylation of the VNTR structure. The 7× VNTR MUC-1 construct (JNW656) produces a more focused smear, centred around ˜65 kDa, consistent with the predicted molecular weight (61 kDa) and heterogeneous glycosylation of the VNTR structure. The 1× VNTR MUC-1 construct (JNW332) produces a faint, single band of ˜40 kDa, consistent with the presence of only a single VNTR unit.

4.5 In Vitro Expression of MUC-1 Constructs—Flow Cytometry

Following transient transfection of the MUC-1 constructs into CHO cells, the expression of MUC-1 on the cell surface was assessed by flow cytometry using the MUC-1 VNTR specific antibody ATR1. The percentage of MUC-1 positive cells was 9.6% for samples transfected with FL-MUC1 (JNW358), 8.8% for samples transfected with 7× VNTR MUC-1 and 9.8% for samples transfected with 1× VNTR MUC-1 (JNW332). This data suggests that the number of VNTRs does not affect the ability of MUC-1 to be translocated to the cell surface and detected by the antibody ATR1.

4.6 Antibody Responses to FL-MUC1, 7× VNTR MUC-1 and 1× VNTR MUC-1 Following PMID Immunisation

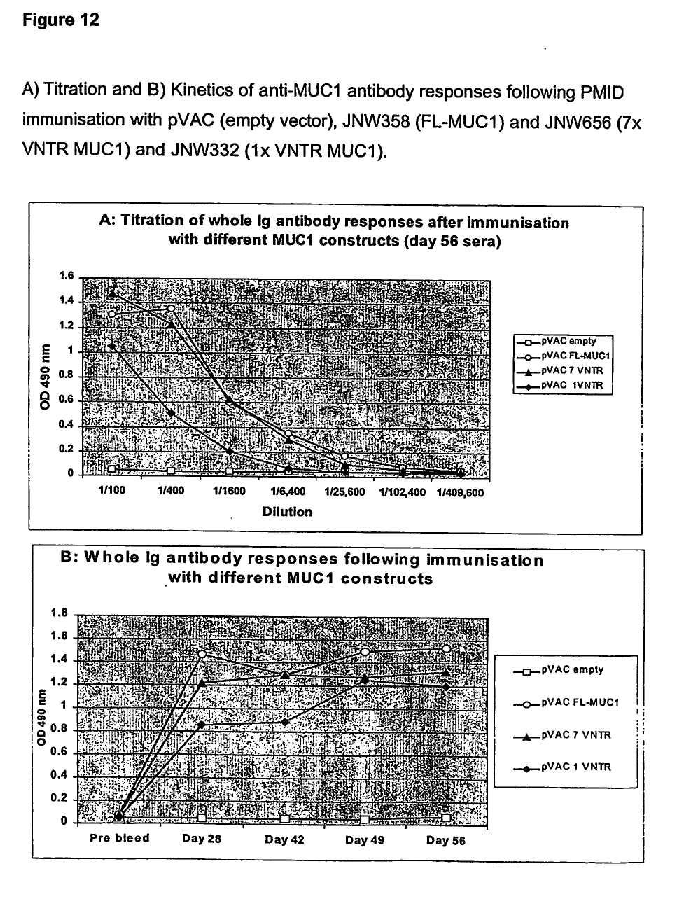

The antibody responses following immunisation with pVAC (empty vector), JNW358 (FL-MUC1), JNW656 (7× VNTR MUC-1) and JNW332 (1× VNTR MUC-1) were assessed by ELISA following a primary immunisation by PMID at day 0 and two boosts at day 21 and day 42. FIG. 12 shows the antibody responses from sera taken at day 56. Whilst there were no MUC1-specific responses to the empty vector, the FL-MUC1 construct and the 7× VNTR-MUC-1 construct produced robust and comparable titres of MUC1-specific antibodies. In contrast, the 1× VNTR MUC-1 construct induced a lower titre antibody response. FIG. 12b shows that the kinetics of the antibody response to FL-MUC1 and 7× VNTR MUC-1 are also very similar, whilst the response to 1× VNTR MUC-1 is slower to develop and requires a second boost at day 42 to reach a plateau. This data confirms that the deletion of a majority of the VNTR units is not detrimental to the induction of a strong, MUC-1-specific antibody response. However, the antibody response to 1× VNTR MUC-1 is sub-optimal in terms of both the magnitude and kinetics of onset.

4.7 Recognition of MUC-1-expressing Tumour Cells by Sera from MUC-1 Immunised Mice

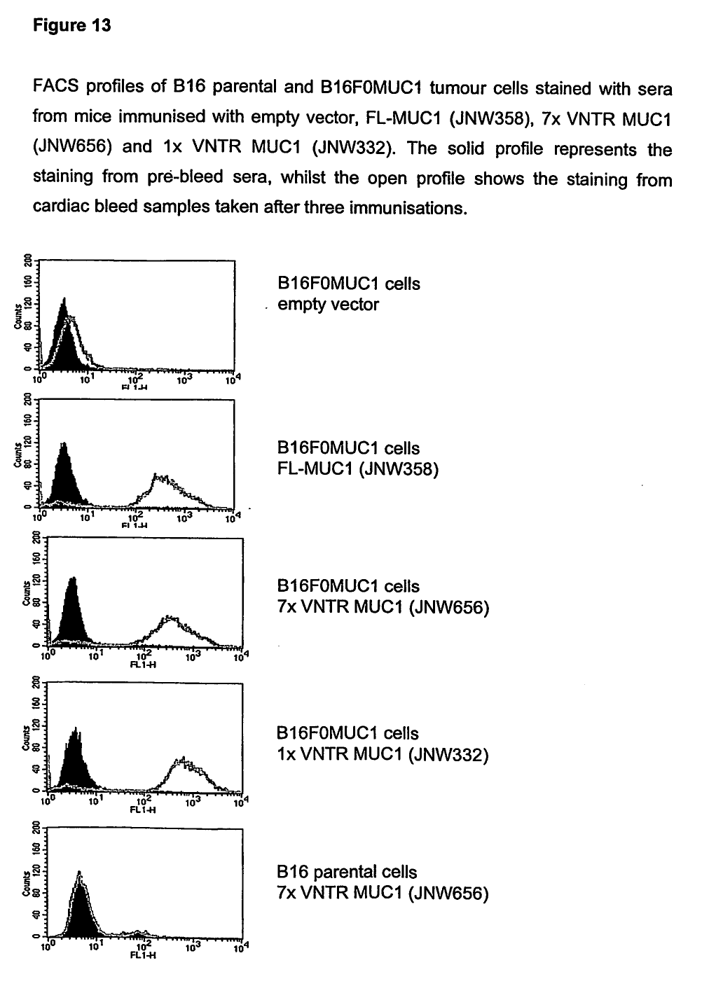

To confirm that the antibodies induced by FL-MUC1, 7× VNTR MUC-1 and 1× VNTR MUC-1 are capable of recognising the form of human MUC-1 expressed on tumour cells, the sera from immunised mice was tested by flow cytometry. The target cells were B16F0MUC1, a tumour cell line which have been engineered to express human MUC-1. The results, shown in FIG. 13, confirm that the sera from FL-MUC1 immunised mice (JNW358), 7× VNTR MUC-1 immunised mice (JNW656) and 1× VNTR MUC-1 immunised mice (JNW332) are equivalent in their ability to recognise MUC-1 expressed on B16F0MUC1, suggesting that the removal of a large number of VNTR units is not detrimental for the induction of a physiologically relevant antibody response.

4.8 Identification of Novel T Cell Epitopes from MUC-1 in C57BL/6 Mice by Screening of a MUC-1 Peptide Library

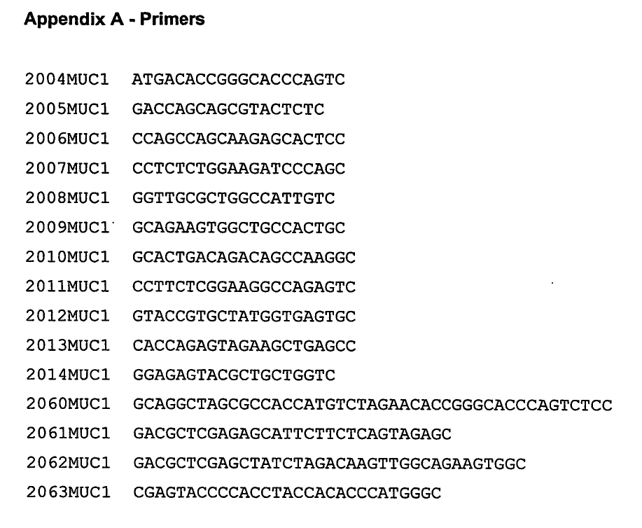

Following immunisation with JNW358 (FL-MUC1) by PMID at day 0 and two boosts at day 21 and day 42, ELISPOT assays were carried out at day 49. Peptides from the FL-MUC1 library were tested at 10 μM final concentration. From this initial screen several groups of 15 mer peptides were found to stimulate IFNγ or IL-2 secretion. The regions of interest are marked on FIG. 20. Peptides stimulating IFNγ secretion were studied further by intracellular cytokine staining and flow cytometry to determine whether the regions contained CD4 or CD8 epitopes. Peptides 223, 224, 225, 238 and 239 were found to induce good IFNγ secretion from CD8 cells. In order to map the CD8 epitopes further, 8 and 9 mer peptides overlapping by either 7 or 8 amino acids were obtained. These were tested in the IFNγ ELISPOT assay and subsequently several showing reactivity were tested by flow cytometry. Region 223-225 contained clusters of CD8 epitopes. By titration, the dominant peptide was shown to be SAPDNRPAL, a peptide that has already been used by others to measure MUC-1 specific responses. However, several novel peptides in this region were identified which induced IFNγ secretion by CD8 cells at 10 μM and lower. We have shown that one of these, TSAPDNRPA is capable of inducing cytotoxic T cells in vitro (data not shown). Region 238-239 was shown to contain one strong CD8 epitope, PTTLASHS, which we have used for subsequent MUC-1 assays, and also several weaker CD8 epitopes.

4.9 Cellular Responses to FL-MUC1, 7× VNTR MUC-1 and 1VNTR MUC-1 Following PMID Immunisation

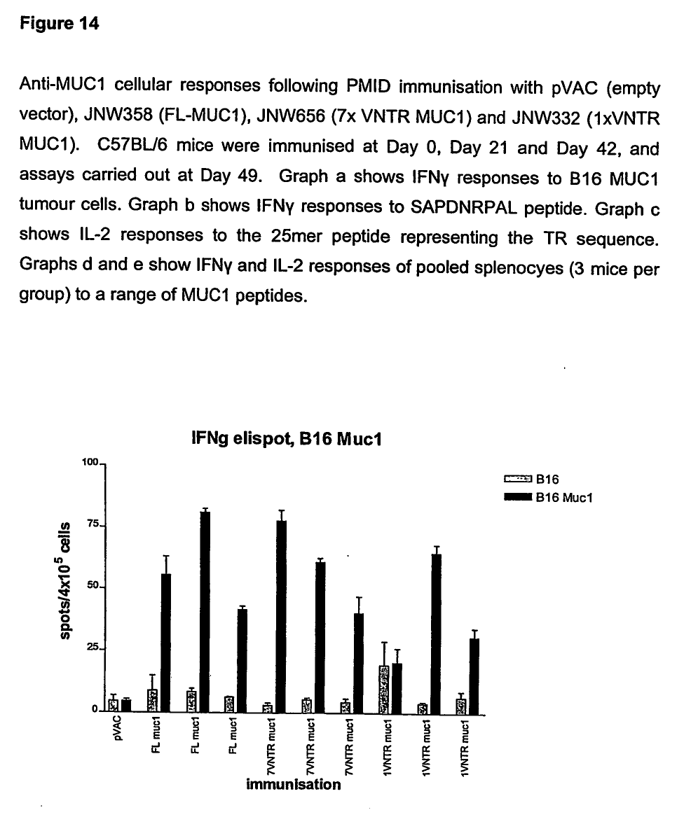

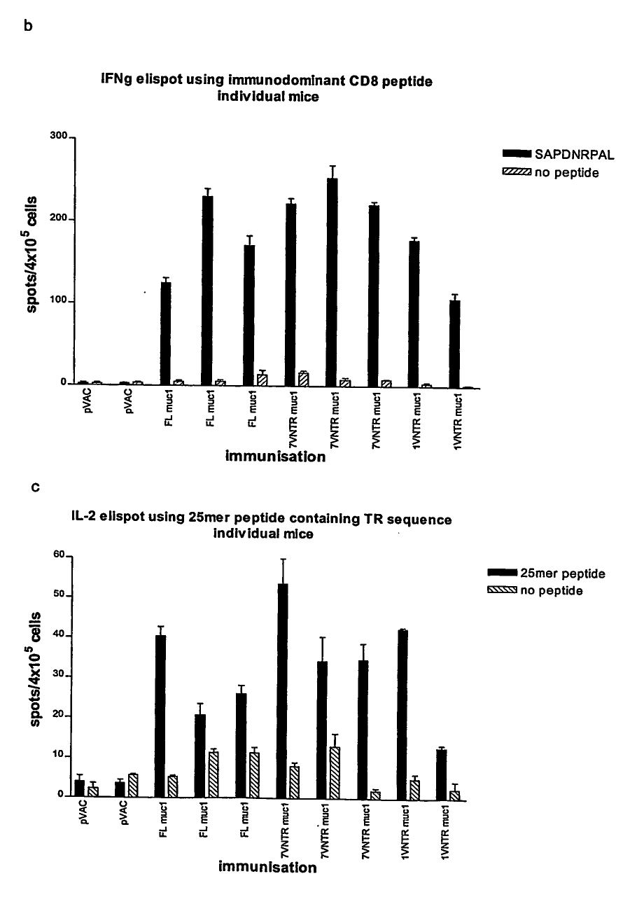

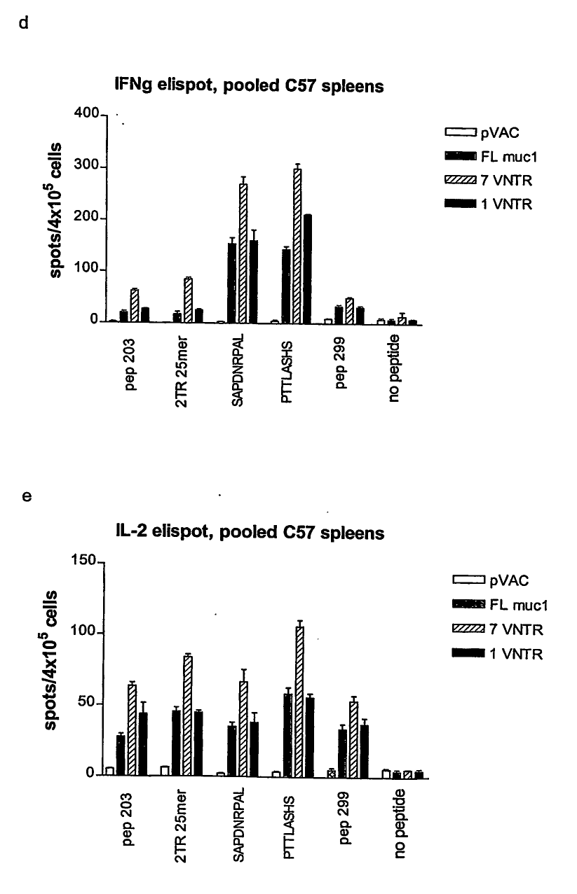

The cellular responses following immunisation with pVAC (empty vector), JNW358 (FL-MUC1), JNW656 (7× VNTR MUC-1) and JNW332 (1× VNTR MUC-1) were assessed by ELISPOT following a primary immunisation by PMID at day 0 and two boosts at day 21 and day 42. Assays were carried out 7 days post boost. Three different assay conditions were used: 1) MUC-1 expressing tumour cells, B16-MUC1 and EL4-MUC1 which are used to demonstrate a broad anti-tumour cellular response, 2) SAPDNRPAL peptide, a high affinity peptide outside the VNTR region of MUC-1 (represented once in the all—constructs used), 3) 25 mer peptide encoding a sequence which includes an entire repeat from the VNTR region and a further 5 amino acids from an adjacent repeat. This peptide induces predominantly IL-2 production from immunised splenocytes. The FL-MUC1 construct, the 7× VNTR-MUC1 and the 1× VNTR-MUC1 construct produced robust and comparable MUC-1-specific cellular responses to all the stimuli tested. FIG. 14). In the case of SAPDNRPAL peptide we have shown that the IFNγ is produced by CD8 cells, while IFNγ production in response to tumour cells and IL-2 production in response to 25 mer peptides may be from either CD4 or CD8 cells. This data confirms that the deletion of a majority of the VNTR units is not detrimental to the induction of a strong, MUC-1-specific cellular response to epitopes either inside or outside the VNTR region.

4.10 Comparison of Protection (PMID vs I.M) Following Tumour Challenge

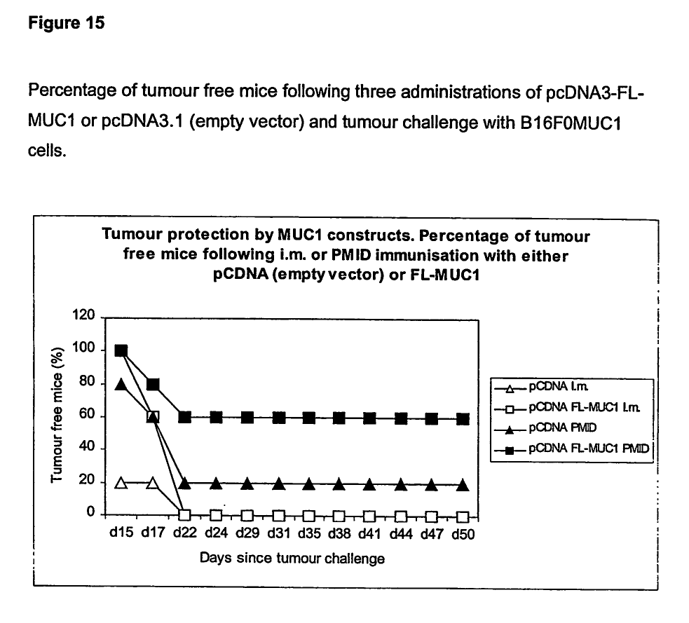

Following three administrations of the MUC-1 expression plasmid pcDNA3-FL-MUC-1 or the empty vector pcDNA3.1, by either PMID or intramuscular injection, mice were challenged with MUC-1-expressing tumour cells (B16F0MUC1). The percentage of tumour free mice is shown in FIG. 15 clearly demonstrating that PMID induces protection from subsequent tumour challenge in a greater number of mice compared to delivery of the same plasmid by intramuscular injection. This data, in conjunction with the antibody and cellular responses detailed above, suggests that PMID induces more robust cellular and antibody responses than intramuscular delivery, correlating with an improved tumour protection profile.

4.11 Efficacy of MUC-1 cDNA Constructs (F/L MUC-1 and 7 VNTR) in Tumour Protection

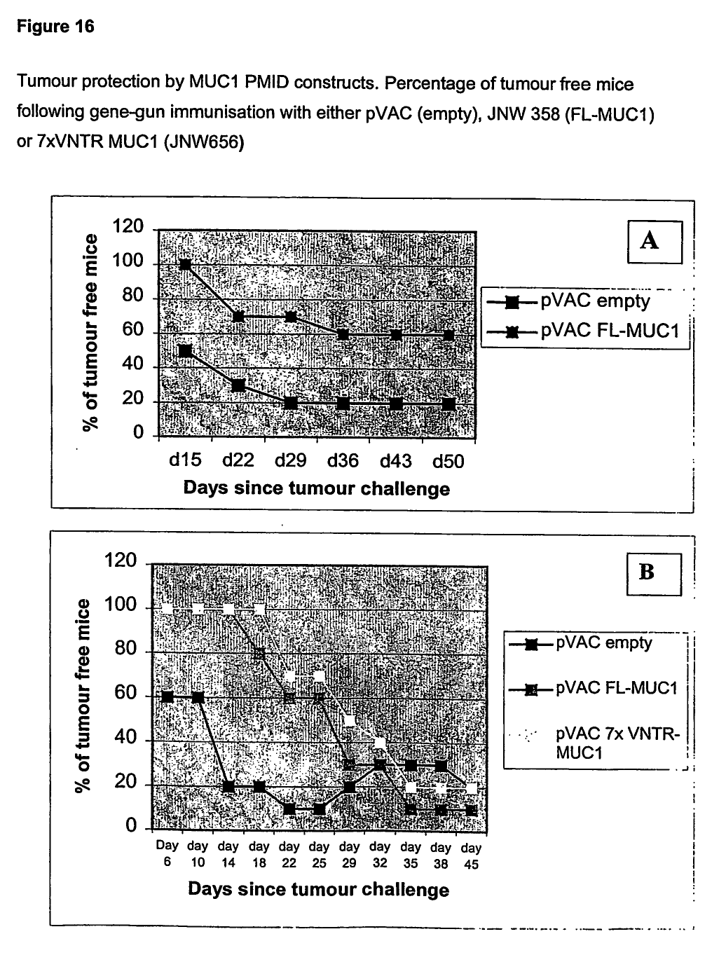

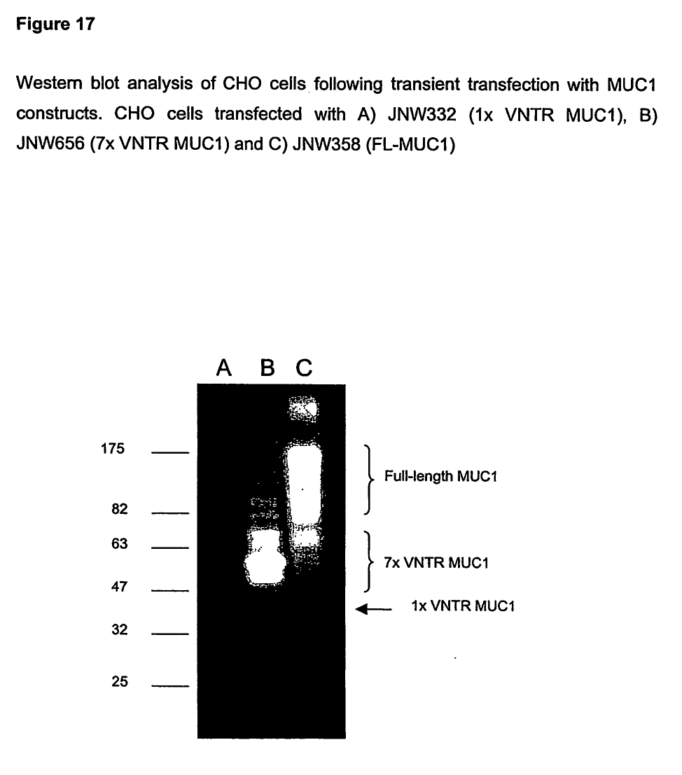

Mice were immunised three times as described in Material and Methods with either the empty vector (pVAC empty) or the vector encoding full-length MUC-1 (JNW358). Two weeks after the last boost they were tumour challenged with B16F0MUC1 cells and tumour growth monitored. When tumours, they appeared approximately 10-15 days after tumour challenge in the empty-vaccinated group and approximately 22 days in the FL-MUC1vaccinated group. FIG. 16a compares the survival of mice immunised with either the empty vector or the vector encoding full-length MUC-1 in both groups. There is a significantly better survival in mice immunised with FL-MUC1 (60% tumour-free) to that in mice immunised with the empty vector (20% tumour-free). FIG. 16b shows the tumour protection comparing both FL-MUC1 and 7× VNTR to the control group with 2× the amount of tumour cells (1.0×106) than in the previous experiments. Both MUC-1 constructs generate a remarkable and comparable delay on tumour growth related to the control vaccinated group until approximately day 25. Later on this effect was decreased, probably due to exhaustion of the immune response to the tumour antigen.

In conclusion 7 VNTR×MUC-1 construct gave the same protective anti-tumour response as FL-MUC-1 even in highly stringent conditions.

4.12 FL vs. 7VNTR MUC-1 Stability in Recombinant Vaccinia Virus System.

Full-length human MUC-1 was inserted into the vector pSClinker as a BamHI fragment. This construct was used to create recombinant vaccinia virus by homologous recombination of the vector into the TK (thymidine kinase) gene of the vaccinia virus genome.

The recombinant virus was plated onto a cell sheet of HTK-cells and the plaques were assayed for beta-galactosidase activity by a bluo-gal assay. The beta-gal gene is carried in the vector and thus blue plaques indicate recombinant virus. A number of blue plaques were selected and cloned until 100% of plaques produced a blue staining when a bluo-gal assay was performed.

6 of these clones were used to infect HTK-cells at a multiplicity of infection of 10 and cells harvested at 6 hr, 24 hr and 32 hr post infection. The cells were resuspended in 200 ul of media and 40ul removed and mixed with SDS-PAGE loading buffer.

These cell extracts were electrophoresed on an SDS PAGE gel and analysed by western blot using the monoclonal antibodies ATR1 and HMFG1 which both recognise epitopes within the VNTR region of MUC-1. None of the recombinant virus infected samples gave any staining with these antibodies. A control cell extract of cells transfected with pVAC-7VNTRMUC1 stained with a bright band indicting presence of TR epitopes. Staining with an anti beta-galactosidase antibody indicated expression of beta-gal in all samples infected with the recombinant virus but not wt virus or cell control.

A molecular analysis of the harvested infected cells was performed by PCR. Primer pairs were chosen which would indicate the presence of the various portions of the pSC11linker-FLMUC1 construct within the recombinant virus genome. The following primer pairs were chosen:

FMC101+2014MUC1—Junction between vector and 5′ end of MUC-1

2008MUC1+FMC102—Junction between vector and 3′ end of MUC-1

2004MUC1+2014MUC1—Portion of MUC-1 5′ of VNTR region

2007MUC1+2009MUC1—Portion of MUC-1 3′ of VNTR region

FMC101 and FMC102 are primers in the vector sequence, which lie 5′ and 3′ respectively, to the linker sequence.

| | |
|---|---|
| FMC101: | CATAAATAATAAATACAATAATTAATTTCTCG |
| FMC102: | GCCTCCTTAAAGCATTTCATACACACAGC |

The 4 PRC reactions shown above were performed using 1 ul of harvested recombinant virus infected cells (32 hr post infection) after heating to 80 deg C. for 10 mins. Reactions were also carried out on samples of wt virus infected cells and non-infected cells. A positive control of 1 ng of pSClinker-FLMUC1 plasmid DNA was also included.

The positive control produced amplified fragments of the correct size when electrophoresed on an agarose gel. None of the other samples produced specific products suggesting that the construct was no longer intact within the viral genome.

Subsequently, a recombinant virus containing a 7VNTR version of human MUC-1 was produced in a similar manner and, after ensuring a clonal population, was used to infect HTK-cells which were harvested as before. Cell extracts of these infected cells clearly demonstrated expression of MUC-1 by western blot with ATR1 and also by FACS analysis of infected cells two days post infection. Mouse MC57 cells infected with 7VNTR recombinant virus were used to stimulate spleen cells from MUC-1 vaccinated mice in an ELISPOT assay. After overnight incubation the spleen cells were shown to secrete IL-2 in response to the 7VNTR vaccinia infected cells but not to wt vaccinia infected cells.

These results suggest that using a MUC-1 construct with 7 tandem repeats improves stability of the construct. The fact that the full-length MUC-1 recombinant vaccinia virus was unable to induce expression of MUC-1 in infected cells strongly suggests that the construct is unstable in this highly recombinogenic setting. None of the 6 virus clones expressed MUC-1 nor did they seem to contain the MUC-1 gene, yet all expressed Beta-Galactosidase, which was carried on the same vector. However, the 7VNTR version with fewer repeats clearly demonstrated expression in 3 different assays indicting greater stability, with no loss of recognition by either antibody or antigen specific T-cells.

5. Stability of FL MUC, 7 VNTR and 1 VNTR when Grown in E. Coli DH1

The relevant vector was used to transform E. Coli DH1. The empty vector was also transformed as a control.

In order to determine if the number of repeats in the VNTR region influences the stability, a shaker flask stability assay was performed using the FL-MUC1, 7× VNTR MUC1 and 1× VNTR MUC1 plasmid.

The stability study looked at the growth, plasmid production and plasmid retention of each of the constructs in shake flask culture over the course of 9 passages, each lasting between 10-14 hr. The use of a stability study is employed in order to determine whether plasmid production and quality changes as a result of the repeated sub-culturing of the cells in shake flasks. As conditions in the shake flasks are uncontrolled (e.g. pH, aeration) the maintenance of plasmid quality and production over the study is a good indication that these characteristics will remain stable.

5. Results

5.2.1. Growth of Cultures

Although there was some variation between the final OD600 nm achieved by the cells from each passage due to slight variations in the inoculum volume, overall there were no significant differences in the growth rates either during the assay or between the different MUC1 constructs.

5.2.2. Plasmid Production

Plasmid copy number values were obtained from the 1^{st}, 5^{th} and 9^{th} (final) passages. For the full-length construct, PCN decreased by 54% over this period, whereas for the other three constructs it increased by ˜40%. The volumetric yield (mg plasmid/1 culture) remained stable throughout the study for the 7VNTR, whereas it decreased by 64% in the full-length construct. A slight decrease in the volumetric yield was observed in the empty vector (21%) and single VNTR construct (24%) though this was by no means as marked as that seen in the full length construct

5.2.3. Plasmid Retention

Plasmid retention was measured using the replica plating assay remained between 80% and 100% for all constructs throughout the stability study. Furthermore, there were no significant differences between the constructs.

5.2.4. Plasmid Stability

To investigate plasmid stability over the duration of this study, both beginning (day 0 harvest) and the end-point (day 5 harvest) plasmid extracts were made with the aid of Qiagen Mini-prep. Plasmid Extraction spin-columns. These extracts were then analysed by agarose gel electrophoretic separation prior to subsequent Sybr-Gold staining. This Sybr-Gold based staining method is considered particular suitable for analysing plasmid stability because of previous work demonstrating it capable of detecting a lng ‘spike’ recombinant within a 1000 ng sample. The results of the investigation are shown below (see FIG. 6) and from these results, three conclusions were drawn:
1. The 7× VNTR and 1× VNTR constructs contain the expected number of VNTR repeats throughout the experiment with no evidence for instability detected either in the plasmid backbone or in VNTR repeat structure at any time point.
2. The p7313 empty vector used in the stability assay does not have the expected profile and differs from the p7313 Plasmid-Standard.
3. The Full-length Mucl samples taken at the final time-point (day 5; 9^{th} passage) contain trace plasmids of unknown origin.

As a consequence of the discrepancy in p7313 profile as well as the identification of trace plasmid species in the FL-Muc1 construct on day 5, further investigative work was undertaken.

To investigate the observed difference between the p7313 empty vector used in the stability study and that of the Plasmid-Standard, restriction enzyme analysis was undertaken. The results of this analysis reveal that an ˜800 bp region of the p7313 construct containing the BamHI (1926 bp) and the SapI (2422 bp) restriction sites had been deleted. Primers were then designed flanking this region and the plasmid subsequently sequenced. The resulting sequence data confirmed that the region between 1866 and 2589 had been deleted. This region of the plasmid contains the Cer sequence. Because this Cer sequences is used to aid resolution of concatemers, its absence may explain the multiple-banding p7313 plasmid species observed in the stability study

Further Investigative Work: Analysis of Trace Plasmids on FL-MUC1 Samples

The trace plasmids observed only in the end-point FL-Muc1 samples were analysed further. This analysis revealed that these traces could not be detected before day 4. Concurrent with this finding, these trace plasmids were also gel-purified, re-transformed, re-purified and sequenced. By such analysis, these plamsids were subsequently identified as contaminants rather than recombinants; these being namely the 7× VNTR used in the stability assay (p7656), the p7313 deleted in the Cer region (outlined above) and also a presumed concatemer of this p7313 Cer deletion.

From these results, it was concluded that FL-MUC1 samples were contaminated with both the p7313 empty vector and 7× VNTR (p7656) constructs and that no recombinants are present in the end-point samples of the FL-Muc1. These contaminants are believed to have entered the FL-Muc1 stock E. coli DH1 during the original transformation of the plasmids. Because the trace plasmids do not appear on agarose gels prior to day 4, one possibility is that they were selected for over course of the study because of their smaller size relative to FL-Mucl plasmid.

3. Conclusion

Data from the stability study showed that the plasmid, 7× VNTR MUC1 is stable in terms of growth characteristics, plasmid retention and plasmid quality. In terms of growth characteristics, plasmid retention and plasmid quality there was no discernible difference between the 1× VNTR, 7× VNTR and the FL-MUC1 vector. However, the copy number data did highlight that there were significant differences between these constructs. Plasmid copy number and volumetric yield both decreased significantly for the full length construct over the course of the stability study, compared to the 7× VNTR. Although plasmid retention was seen to remain at 100% throughout the experiment for the full-length construct, this only indicates that all cells in the population still contain sufficient plasmid to confer kanamycin resistance on them. If the experiment was to be run for longer it is possible that the copy number could decrease to such a level that the resistance to kanamycin would not be sufficient to enable growth on selective plates, resulting in a decrease in observed plasmid retention. These data suggest that the 7× VNTR construct may possess significant advantages in terms of a favourable development profile since. The plasmid content can have an effect on the purification of the cell paste. With the differences between the 7VNTR and FL-MUC1 construct, it is likely that 7 VNTR will be easier to purify and to be obtained in higher yields.

1. A nucleic acid molecule encoding a MUC-1 antigen, said molecule being capable of raising an immune response in vivo and having reduced susceptibility to recombination than full-length MUC-1.

2. A nucleic acid molecule encoding a MUC-1 antigen comprising between 1 and 15 VNTR perfect repeat units.

3. A nucleic acid molecule as claimed in claim 2 comprising less than 8 perfect VNTR repeat units.

4. A nucleic acid molecule as claimed in claim 1 wherein at least one VNTR is mutated to reduce the potential for glycosylation.

5. A nucleic acid molecule as claimed in claim 1 that incorporates a sequences encoding an epitope selected from the group: FLSFHITSNL, NSSLEDPSTDYYQELQRDISE and NLTISDVSV.

6. A nucleic acid molecule as claimed in claim 1 wherein the molecule is a DNA molecule.

7. A plasmid comprising the DNA molecule of claim 6.

8. A protein encoded by a nucleic acid as claimed claim 1.

9. A pharmaceutical composition comprising a nucleic acid as claimed in claim 1 and a pharmaceutical acceptable excipient, diluent or carrier.

10. A pharmaceutical composition as claimed in claim 9 wherein the carrier is microparticle.

11. A pharmaceutical composition as claimed in claim 10 wherein the microparticle is gold.

12. A pharmaceutical composition as claimed in claim 9 comprising an adjuvant.

13. A nucleic acid as claimed in claim 1 for use in medicine.

14. Use of a nucleic acid as claimed in claim 1 the preparation of a medicament for the treatment or prevention MUC-1 expressing tumours.

15. Use of a protein as claimed in claim 8 in the manufacture of a medicament for the treatment or prevention of MUC-1 expressing tumours.

16. A method of treating or preventing tumours or metastases, comprising administering a safe and effective amount of a nucleic acid as claimed in claim 1.


SEQUENCE LISTING (24 sequences)

SEQ ID NO: 1 - protein, 20 aa, Human
APDTRPAPGSTAPPAHGVTS

SEQ ID NO: 2 - protein, 20 aa, human
APDTRPAPGSTAPPAHGVTS

SEQ ID NO: 3 - protein, 20 aa, human
APATEPASGSAATWGQDVTS

SEQ ID NO: 4 - protein, 20 aa, human
VPVTRPALGSTTPPAHDVTS

SEQ ID NO: 5 - protein, 20 aa, human
APDNKPAPGSTAPPAHGVTS

SEQ ID NO: 6 - protein, 20 aa, human
APDNRPALGSTAPPVHNVTS

SEQ ID NO: 7 - protein, 9 aa, Artificial Sequence
  Rat
SAPDNRPAL

SEQ ID NO: 8 - protein, 25 aa, Artificial Sequence
  Rat
PAHGVTSAPDTRPAPGSTAPPAHGV

SEQ ID NO: 9 - protein, 15 aa, Artificial Sequence
  Rat
DVTLAPATEPATEPA

SEQ ID NO: 10 - protein, 15 aa, Artificial Sequence
  Rat
LSYTNPAVAATSANL

SEQ ID NO: 11 - protein, 8 aa, Artificial Sequence
  Rat
PTTLASHS

SEQ ID NO: 12 - DNA, 32 bp, Artificial Sequence
  PCR primer
cataaataataaatacaataattaatttctcg

SEQ ID NO: 13 - DNA, 29 bp, Artificial Sequence
  PCR primer
gcctccttaaagcatttcatacacacagc

SEQ ID NO: 14 - protein, 9 aa, human
FLSFHISNL

SEQ ID NO: 15 - protein, 21 aa, human
NSSLEDPSTDYYQELQRDISE

SEQ ID NO: 16 - protein, 9 aa, human
NLTISDVSV

SEQ ID NO: 17 - DNA, 1774 bp, human
ggatccgctccacctctcaagcagccagcgcctgcctgaatctgttctgccccctccccacccatttcaccaccaccatgacaccgggcacccagtctcctttcttcctgctgctgctcctcacagtgcttacagttgttacaggttctggtcatgcaagctctaccccaggtggagaaaaggagacttcggctacccagagaagttcagtgcccagctctactgagaagaatgctgtgagtatgaccagcagcgtactctccagccacagccccggttcaggctcctccaccactcagggacaggatgtcactctggccccggccacggaaccagcttcaggttcagctgccacctggggacaggatgtcacctcggtcccagtcaccaggccagccctgggctccaccaccccgccagcccacgatgtcacctcagccccggacaacaagccagccccgggctccaccgcccccccagcccacggtgtcacctcggccccggacaccaggccggccccgggctccaccgcccccccagcccatggtgtcacctcggccccggacaacaggcccgccttgggctccaccgcccctccagtccacaatgtcacctcggcctcaggctctgcatcaggctcagcttctactctggtgcacaacggcacctctgccagggctaccacaaccccagccagcaagagcactccattctcaattcccagccaccactctgatactcctaccacccttgccagccatagcaccaagactgatgccagtagcactcaccatagcacggtacctcctctcacctcctccaatcacagcacttctccccagttgtctactggggtctctttctttttcctgtcttttcacatttcaaacctccagtttaattcctctctggaagatcccagcaccgactactaccaagagctgcagagagacatttctgaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtaggggcacgtcgcccgctgagctgagtggccagccagtgccattccactccactcaggttcttcagggccagagcccctgcaccctgtttgggctggtgagctgggagttcaggtgggctgctcacaccgtccttcagaggccccaccaatttctcggacacttctcagtgtgtggaagctcatgtgggcccctgaggctcatgcctgggaagtgttgtggtgggggctcccaggaggactggcccagagagccctgagatagcggggatcc

SEQ ID NO: 18 - protein, 475 aa, human
MTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSTVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 19 - DNA, 1457 bp, human
gctagcgccaccatgtctagaacaccgggcacccagtctcctttcttcctgctgctgctcctcacagtgcttacagttgttacaggttctggtcatgcaagctctaccccaggtggagaaaaggagacttcggctacccagagaagttcagtgcccagctctactgagaagaatgctgtgagtatgaccagcagcgtactctccagccacagccccggttcaggctcctccaccactcagggacaggatgtcactctggccccggccacggaaccagcttcaggttcagctgccacctggggacaggatgtcacctcggtcccagtcaccaggccagccctgggctccaccaccccgccagcccacgatgtcacctcagccccggacaacaagccagccccgggctccaccgcccccccagcccacggtgtcacctcggccccggacaccaggccggccccgggctccaccgcccccccagcccatggtgtcacctcggccccggacaacaggcccgccttgggctccaccgcccctccagtccacaatgtcacctcggcctcaggctctgcatcaggctcagcttctactctggtgcacaacggcacctctgccagggctaccacaaccccagccagcaagagcactccattctcaattcccagccaccactctgatactcctaccacccttgccagccatagcaccaagactgatgccagtagcactcaccatagcacggtacctcctctcacctcctccaatcacagcacttctccccagttgtctactggggtctctttctttttcctgtcttttcacatttcaaacctccagtttaattcctctctggaagatcccagcaccgactactaccaagagctgcagagagacatttctgaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtctagatagctcgag

SEQ ID NO: 20 - protein, 479 aa, human
MSRTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSTVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANLSR

SEQ ID NO: 21 - DNA, 2135 bp, human
ggatccgctccacctctcaagcagccagcgcctgcctgaatctgttctgccccctccccacccatttcaccaccaccatgacaccgggcacccagtctcctttcttcctgctgctgctcctcacagtgcttacagttgttacaggttctggtcatgcaagctctaccccaggtggagaaaaggagacttcggctacccagagaagttcagtgcccagctctactgagaagaatgctgtgagtatgaccagcagcgtactctccagccacagccccggttcaggctcctccaccactcagggacaggatgtcactctggccccggccacggaaccagcttcaggttcagctgccacctggggacaggatgtcacctcggtcccagtcaccaggccagccctgggctccaccaccccgccagcccacgatgtcacctcagccccggacaacaagccagccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggcccgccccgggctccaccgcccccccagcccacggtgtcacctcggccccggacaccaggcccgccccgggctccaccgcgcccgcagcccacggtgtcacctcggccccggacaccaggccggccccgggctccaccgccccccaagcccacggtgtcacctcggccccggacaccaggccggccccgggctccaccgcccccccagcccatggtgtcacctcggccccggacaacaggcccgccttgggctccaccgcccctccagtccacaatgtcacctcggcctcaggctctgcatcaggctcagcttctactctggtgcacaacggcacctctgccagggctaccacaaccccagccagcaagagcactccattctcaattcccagccaccactctgatactcctaccacccttgccagccatagcaccaagactgatgccagtagcactcaccatagcacggtacctcctctcacctcctccaatcacagcacttctccccagttgtctactggggtctctttctttttcctgtcttttcacatttcaaacctccagtttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtaggggcacgtcgcccgctgagctgagtggccagccagtgccattccactccactcaggttcttcagggccagagcccctgcaccctgtttgggctggtgagctgggagttcaggtgggctgctcacaccgtccttcagaggccccaccaatttctcggacacttctcagtgtgtggaagctcatgtgggcccctgaggctcatgcctgggaagtgttgtggtgggggctcccaggaggactggcccagagagccctgagatagcggggatcc

SEQ ID NO: 22 - protein, 595 aa, human
MTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPAAHGVTSAPDTRPAPGSTAPQAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSTVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 23 - DNA, 1835 bp, human
ggatccgctccacctctcaagcagccagcgcctgcctgaatctgttctgccccctccccacccatttcaccaccaccatgacaccgggcacccagtctcctttcttcctgctgctgctcctcacagtgcttacagttgttacaggttctggtcatgcaagctctaccccaggtggagaaaaggagacttcggctacccagagaagttcagtgcccagctctactgagaagaatgctgtgagtatgaccagcagcgtactctccagccacagccccggttcaggctcctccaccactcagggacaggatgtcactctggccccggccacggaaccagcttcaggttcagctgccacctggggacaggatgtcacctcggtcccagtcaccaggccagccctgggctccaccaccccgccagcccacgatgtcacctcagccccggacaacaagccagccccgggctccaccgcccccccagcccacggtgtcacctcggccccggacaccaggccggccccgggctccaccgccccccaagcccacggtgtcacctcggccccggacaccaggccggccccgggctccaccgcccccccagcccatggtgtcacctcggccccggacaacaggcccgccttgggctccaccgcccctccagtccacaatgtcacctcggcctcaggctctgcatcaggctcagcttctactctggtgcacaacggcacctctgccagggctaccacaaccccagccagcaagagcactccattctcaattcccagccaccactctgatactcctaccacccttgccagccatagcaccaagactgatgccagtagcactcaccatagcacggtacctcctctcacctcctccaatcacagcacttctccccagttgtctactggggtctctttctttttcctgtcttttcacatttcaaacctccagtttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtaggggcacgtcgcccgctgagctgagtggccagccagtgccattccactccactcaggttcttcagggccagagcccctgcaccctgtttgggctggtgagctgggagttcaggtgggctgctcacaccgtccttcagaggccccaccaatttctcggacacttctcagtgtgtggaagctcatgtgggcccctgaggctcatgcctgggaagtgttgtggtgggggctcccaggaggactggcccagagagccctgagatagcggggatcc

SEQ ID NO: 24 - protein, 495 aa, human
MTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPQAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSTVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL